SEQUENCE LISTING (2 sequences)

SEQ ID NO: 1 - DNA, 687 bp, Homo sapiens
atgagttatgcagaaaaacccgatgaaatcacgaaagatgagtggatggaaaagctcaataacttgcatgtccagagagcagacatgaaccgcctcatcatgaactacctggtcacagagggctttaaggaagcagcggagaagtttcgaatggaatctggaatcgaacctagtgtggatctggaaacacttgatgaacgaatcaagatccgggagatgatactgaaaggtcagattcaggaggccatcgccttgatcaacagcctccacccagagctcttggacacaaaccggtatctttacttccatttgcagcaacagcatttgatcgagctgatccgccagcgggagacagaggcggcgctggagtttgcacagactcagctggcggagcagggcgaggagagccgagagtgcctcacagagatggagcgtaccctggcactgctggcctttgacagtcccgaggagtcgcccttcggagacctcctccacaccatgcagaggcagaaggtgtggagtgaagttaaccaagctgtgctagattatgaaaatcgcgagtcaacacccaaactggcaaaattactgaaactactactttgggctcagaacgagctggaccagaagaaagtaaaatatcccaaaatgacagacctcagcaagggtgtgattgaggagcccaagtag

SEQ ID NO: 2 - protein, 228 aa, Homo sapiens
MSYAEKPDEITKDEWMEKLNNLHVQRADMNRLIMNYLVTEGFKEAAEKFRMESGIEPSVDLETLDERIKIREMILKGQIQEAIALINSLHPELLDTNRYLYFHLQQQHLIELIRQRETEAALEFAQTQLAEQGEESRECLTEMERTLALLAFDSPEESPFGDLLHTMQRQKVWSEVNQAVLDYENRESTPKLAKLLKLLLWAQNELDQKKVKYPKMTDLSKGVIEEPK

(19) United States
(12) Patent Application Publication
(10) Pub. No.: US 2020/0326343 A1
(43) Pub. Date: Oct. 15, 2020

(54) GENE AND ITS EXPRESSION PRODUCT PROMOTING THE OCCURRENCE AND DEVELOPMENT OF CANCER AND APPLICATION

(71) Applicant: Zhejiang University, Hangzhou (CN)

(72) Inventors: Tianhua ZHOU, Hangzhou (CN); Yi LU, Hangzhou (CN); Yuehong YANG, Hangzhou (CN); Qiong LIU, Hangzhou (CN); Yaoping HUANG, Hangzhou (CN); Shanshan XIE, Hangzhou (CN)

(21) Appl. No.: 16/794,240

(22) Filed: Feb. 19, 2020

Related U.S. Application Data
(63) Continuation of application No. PCT/CN2018/101207, filed on Aug. 17, 2018.

(30) Foreign Application Priority Data
Aug. 20, 2017 (CN) 201710715813.2
Aug. 17, 2018 (CN) 201810937904.5

(51) Int. Cl. G01N 33/574; C07K 16/30

(52) U.S. Cl. CPC G01N 33/574; C07K 16/30

(57) ABSTRACT

The present disclosure discloses the Twa1 gene and its expression products and applications. This disclosure first discovers and verifies the role of the Twa1 gene and its expression product in the occurrence and development of cancer. The Twa1 gene and its expression product has the ability to promote cell proliferation, growth, migration, invasion, and tumor formation, thereby providing a diagnosis of malignant tumors. This disclosure also provides new targets and strategies for inhibiting or interfering with the occurrence and development of many malignant tumors. The present disclosure provides an expression vector containing the Twa1 gene and a transgenic cell line and host bacteria containing the expression vector.

### CROSS REFERENCE TO RELATED APPLICATIONS

This application is a continuation of PCT Patent Application No. PCT/CN2018/101207, filed on Aug. 17, 2018, entitled “GENE AND ITS EXPRESSION PRODUCT PROMOTING THE OCCURRENCE AND DEVELOPMENT OF CANCER AND APPLICATION,” which claims foreign priority of Chinese Patent Application No. 201710715813.2, filed Aug. 20, 2017 and No. 201810937904.5, filed Aug. 17, 2018, in the China National Intellectual Property Administration, the entire contents of which are hereby incorporated by reference in their entireties.

### SEQUENCE LISTING

This application contains a Sequence Listing in form of ASCII text filed via EFS-Web and hereby incorporated by reference into the specification in its entirety. The name of the text file containing the Sequence Listing is 17-CGZD1020003-SequenceListing. The size of the text file is 3250 bytes, and the text file was created on Feb. 19, 2020.

### TECHNICAL FIELD

The present disclosure belongs to the technical field of oncology and gene therapy. Specifically, the present disclosure provides a gene and its expression product which have a promoting effect on tumorigenesis and cancer development, and the application.

### BACKGROUND

Twa1 (Two hybrid associated protein No. 1 with RanBPM), also known as GID8 or C20orf11, is an evolutionarily conserved gene that consists of 687 base pairs and is located on human chromosome 20. Twa1 protein comprises 228 amino acids, localized in both the cytoplasm and the nucleus, and can interact with RanBPM (Ran-binding protein M) protein. Currently, there are only few studies on Twa1. Preliminary studies reported its protein composition and suggested a role in cell migration and embryonic development while the molecular mechanism is unclear. The function of Twa1 in the occurrence and development of cancer has never been elucidated.

Tumor is one of the most serious diseases that threaten human health and life. Tumor (also known as cancer) refers to the neogrowth formed by the over-proliferating cells in local tissue under the stimulation of various tumorigenic factors. This neogrowth is also called neoplasm, because it often displays as a protuberance. The process by which normal cells are transformed into tumor cells is called the occurrence and development of tumors. The occurrence and development of tumors is a progressive process involving the accumulation of multiple mutations. During this process, cancerous cells are no longer controlled by normal regulatory mechanisms and invade adjacent normal tissues. After a malignant transformation, tumor cells continue to accumulate mutations, rendering the mutant cells new characteristics which makes them more dangerous. According to the latest World Health Organization data on global disease, the mortality rate of malignant tumors ranks second. In recent years, the incidence and mortality of tumors have gradually increased. Although the development of medical technology and cancer treatment methods with surgery, radiotherapy and chemotherapy have made progress, finding more effective targets, molecules, and therapeutic drugs is still of great clinical significance because of the complicated mechanism of cancer pathogenesis and extremely difficult treatments.

Taking colorectal cancer as an example, a large number of studies have shown that aberrant activation of the canonical Wnt signaling pathway is one of the major causes of cancer. The Wnt signaling pathway is initiated by the binding of extracellular Wnt ligands to receptors on the cell membrane, which increases the stability of the β-catenin protein in the cytoplasm, promotes β-catenin translocation to the nucleus, and activates the downstream target gene expression. Studies have shown that most colorectal cancer is caused by APC (adenomatous polyposis coli) gene mutation in the Wnt pathway. Mutant APC protein prevents β-catenin degradation in the cytoplasm, leading to β-catenin nuclear translocation and accumulation there. Therefore, downstream target genes (such as cyclin D1 and c-Myc) are constitutively expressed, resulting in cell over proliferation and colorectal tumorigenesis. In addition, mutations in other components of the Wnt signaling pathway, such as Axin loss-of-function mutation and gain-of-function mutation in β-catenin, are also present in colorectal cancer tissue, and these mutations eventually lead to elevated levels of β-catenin in the nucleus, thereby promoting colorectal cancer development. These studies indicate that aberrant accumulation of β-catenin in the nucleus is one of the major causes for colorectal tumorigenesis. Moreover, β-catenin nuclear accumulation is often observed in other malignant tumors, such as breast cancer, liver cancer, and lung cancer. However, the molecular mechanism for β-catenin nuclear accumulation remains poorly understood.

Gene expression is initiated by a transcription factor, which is a group of protein molecules that can specifically bind to the promoter of a gene to ensure the gene is expressed at a specific time and intensity. As a transcriptional coactivator, β-catenin binds to the transcriptional factor and activates the transcription factor to promote gene expression and cell proliferation. In most types of tumors, β-catenin is highly expressed in the nucleus of tumor cells. Current studies have identified some small molecule inhibitors that block the binding between β-catenin and transcription factors in order to inhibit the target gene expression and tumor cell proliferation. However, these small molecule inhibitors have no significant effect on the inhibition of tumor growth, and are not well-applied clinically. One of the reasons is that there is a large amount of β-catenin which can bind to a variety of transcription factors in the nucleus to promote tumor cell proliferation. These small molecule inhibitors can't prevent all β-catenin binding to the transcription factors in the nucleus, and also can't block the binding of β-catenin to all transcription factors. This indicates that the present technologies can't completely inhibit the function of β-catenin in the nucleus, and there is no inhibitor specifically targeting the accumulation of β-catenin in the nucleus. If we can elucidate the molecular mechanism of β-catenin nuclear accumulation, screening and preparation of drugs that can inhibit or interfere with β-catenin nuclear accumulation will most likely inhibit tumor cell growth, as well as the occurrence and development of cancer. Therefore, it is of great theoretical significance and application value to understand the molecular mechanism of β-catenin nuclear accumulation and u new effective targets.

RNA interference (RNAi) refers to the endogenous or exogenous double-stranded RNA (dsRNA)-mediated degradation of intracellular mRNA, resulting in the inhibition of target gene expression and loss of biological function. This phenomenon belongs to a post-transcriptional gene silencing mechanism. siRNA is a 21-nucleotide dsRNA composed of a sense strand nucleotide and an antisense strand nucleotide. The sense strand and the antisense strand nucleotides are complementary paired. Introducing specific siRNA into tumor cells can specifically inhibit the expression of target genes in tumor cells. Therefore, it can be used as a cancer treatment method, which shows a high specificity in compared to traditional surgery, radiotherapy and chemotherapy. This method greatly reduces the damage to normal cells and improves the accuracy of gene therapy. Another way to deliver siRNA in cells is cloning the siRNA sequence as a short hairpin into a plasmid vector. When the vector enters the cell, the hairpin sequence is transcribed to form a short hairpin RNA (shRNA) and further processed to siRNA. The shRNA cloned into the shRNA expression vector consists of two short inverted repeats separated by a stem loop sequence to form a hairpin structure whose expression is controlled by the RNA polymerase III.

The CRISPR/Cas9 is an adaptive immune defense system formed by bacteria and archaea during long-term evolution and used against invading viruses. The principle is that crRNA (CRISPR-derived RNA) binds to tracrRNA (trans-activating RNA) to form a tracrRNA/crRNA complex, and then directs the nuclease Cas9 protein to a target site which is paired with crRNA to cut double-stranded DNA and activates DNA damage repair process in cells. The DNA repair process can cause the effects of INDEL (insertion and deletion), which leads to the frameshift mutation of the gene to achieve the purpose of gene knockout. By designing these two RNAs, a sgRNA (single-guide RNA) can be engineered and cloned into a plasmid vector. When the vector enters the cell, the corresponding sgRNA can be expressed, together with CRISPR/Cas9 system accurately cuts the corresponding target gene. By introducing specific sgRNA into tumor cells to knock out target gene in tumor cells, which can be used as an important cancer treatment.

### SUMMARY OF THE DISCLOSURE

In view of the deficiency of previous technologies, the technical problem solved by the present disclosure is to provide a Twa1 gene and its expression product which have a promoting effect on tumor occurrence and development, and an application. Applicants first discover and verify the role of Twa1 gene in the occurrence and development of cancer by systematically investigating the biological function and molecular mechanism of Twa1 gene. The inventors find that Twa1 gene promotes tumor cell proliferation, migration, and invasion, which provides a new target and idea for the diagnosis and therapy of a variety of malignant tumors. Applicants have successfully cloned the Twa1 gene, and the cDNA sequence of Twa1 (see the DNA sequence of SEQ ID NO. 1 in FIG. 16 of the specification, that is, the gene sequence of human Twa1). Applicants extracted total cellular RNA and transcribed the Twa1 cDNA sequence by using a reverse transcription kit, performed PCR for amplification using specific primers for the Twa1 cDNA sequence, inserted the sequence into a prokaryotic or eukaryotic expression vector (see Embodiment 4), and purified Twa1 polypeptide or protein from prokaryotic or eukaryotic cells (see the sequence of SEQ ID NO. 2 in the sequence listing of the specification, that is, the amino acid sequence of human Twa1, and Embodiment 6).

In some embodiments, applicants have discovered the function of the Twa1 gene in regulating β-catenin nuclear accumulation, providing new targets and ideas for screening and preparing anti-tumor drugs which are capable of inhibiting the nuclear accumulation of β-catenin, thereby effectively suppressing tumorigenesis.

Applicants have also discovered the molecular regulatory mechanism of Twa1 gene in canonical Wnt signaling pathway. Nuclear accumulation of β-catenin is a hallmark of the occurrence and development of various malignant tumors, such as colorectal cancer, breast cancer, liver cancer, glioblastoma, and melanoma. Applicants demonstrate that Twa1 enhances the Wnt signaling pathway by promoting nuclear accumulation of β-catenin, thereby promoting tumorigenesis and progression.

Applicants have further discovered that Twa1 regulates epithelial-mesenchymal transition (EMT). EMT plays a key role not only in developmental process but also in tissue wound healing, organ fibrosis, and tumorigenesis. EMT promotes the metastasis of a variety of malignant tumors, such as colorectal cancer, bladder cancer, liver cancer, and melanoma. Applicants have discovered that the Twa1 gene promotes the migration and invasion of tumor cells by promoting EMT, thereby promoting tumorigenesis and development.

In the present disclosure, the tumor refers to a neogrowth formed by the over-proliferating cells in local tissue due to genetic mutation under the stimulation of various tumorigenic factors. Preferentially, the tumor in the present disclosure can be colorectal cancer, breast cancer, sarcoma, lung cancer, prostate cancer, kidney cancer, pancreatic cancer, blood cancer, neuroblastoma, glioma, head cancer, neck cancer, thyroid gland. cancer, liver cancer, ovarian cancer, vulvar cancer, cervical cancer, endometrial cancer, testicular cancer, bladder cancer, esophageal cancer, stomach cancer, nasopharyngeal cancer, buccal cancer, oral cancer, gastrointestinal stromal tumor, skin cancer, multiple myeloma, glioblastoma, or melanoma.

In order to solve the technical problem of the present disclosure, the present disclosure provides the following technical solutions:

The present disclosure provides an isolated nucleic acid that promotes the occurrence and development of tumor encodes Twa1 gene. The nucleic acid comprises a DNA sequence having at least one of the following characteristics:
1) the DNA sequence of SEQ ID NO. 1 in the Sequence Listing;
2) a polynucleotide encoding the protein sequence of SEQ ID NO. 2 in the Sequence Listing;
3) a nucleotide sequence which hybridizes to a DNA sequence defined by SEQ ID NO. 1 in the Sequence Listing under high stringency conditions;
4) a DNA sequence having 90% or more homology with the DNA sequence of SEQ ID NO. 1 in the Sequence Listing, and encoding the same functional protein.

The DNA sequence of SEQ ID NO. 1 in the Sequence Listing consists of 687 base pairs.

The Twa1 may also refer to GID8 or C20orf11.

The occurrence and development of cancer refers to a process in which normal cells are transformed into tumor cells. The occurrence and development of tumor is a progressive process involving the accumulation of multiple mutations. During this process, cancer cells are uncontrolled by normal regulatory mechanisms and progressively invade adjacent normal tissues. After a malignant transformation of the cells, tumor cells continue to accumulate mutations, rendering the mutant cells new characteristics that make them more dangerous. The Twa1 gene in the present disclosure promotes the occurrence and development of tumor, that is, to promote the transformation of normal cells into tumor cells. When the tumor lacks the Twa1 gene, the occurrence and development of the tumor could be inhibited or interfered. The inhibition refers to completely blocking the occurrence and development of tumors, and the interference refers to affecting the occurrence and development of tumors to varying degrees.

The disclosure provides a polypeptide or protein, also known as a Twa1 polypeptide or protein, which has a promoting effect on the occurrence and development of tumor, and comprises at least one of the following characteristics.

1) The sequence of SEQ ID NO. 2 in the Sequence Listing.

2) A protein which is substituted and/or deleted and/or added by one or several amino acid residues of the amino acid residue sequence of SEQ ID NO. 2 in the Sequence Listing and associated with tumor.

Substitutions and/or deletions and/or additions of one or more amino acid residues refer to substitutions and/or deletions and/or additions of no more than 10 amino acid residues.

3) A polypeptide or protein having 90% or more homology with the amino acid residue sequence of SEQ ID NO. 2 in the Sequence Listing, and associated with a tumor.

The amino acid of SEQ ID NO. 2 in the Sequence Listing consists of 228 amino acid residues.

The Twa1 polypeptide or protein of the present disclosure is expressed by the intracellular transcription or translation process of the Twa1 gene. The transcription refers to a process in which a cell uses the DNA sequence of the Twa1 gene as a template to synthesize a corresponding Twa1 mRNA by using a ribonucleotide as the raw material based on the nucleotide complementary pairing principle. The translation refers to a process in which cells further synthesize a corresponding Twa1 polypeptide or protein using the Twa1 mRNA as template and using amino acids as a raw material. The Twa1 polypeptide or protein promotes the occurrence and development of tumors, that is, to promote the transformation of normal cells into tumor cells. When a tumor lacks the Twa1 polypeptide or protein, the occurrence and development of the tumor will be inhibited or interfered.

The present disclosure provides a gene expression vector which promotes the occurrence and development of tumors, and the vector contains the DNA sequence of the Twa1 gene as described above. The Twa1 gene of the present disclosure can be inserted into an existing prokaryotic or eukaryotic expression vector, and suitable vectors include bacterial plasmids, lentiviruses, adenoviruses, adeno-associated viruses, and retroviruses. The vector is a small circular DNA molecule capable of autonomous replication and transcriptional expression in cells, and is the most commonly used tool in genetic engineering. The expression vector containing Twa1 gene of the present disclosure can be used to transform a suitable cell line or host strain such as the transgenic cell line or host strain to express a Twa1 polypeptide or protein. An antibody is prepared using the Twa1 polypeptide or protein as an antigen. Since the antibody specifically binds to a Twa1 polypeptide or protein, it can be used for detecting the abundance of Twa1 polypeptide or protein in clinical samples such as human body fluid, blood, cells, tissues. The detection method may be a commonly used method, such as immunohistochemistry, immunoblotting, immunofluorescence, and ELISA (enzyme linked immunosorbent assay). According to the abundance of Twa1 polypeptide or protein in the clinical sample, it can be characterized whether the patient who provides the sample has a tumor. If a high level of Twa1 polypeptide or protein is detected in a clinical sample, it indicates that the patient is likely to have a tumor. While a low level of Twa1 polypeptide or protein in a clinical sample is detected, indicating that the patient is likely to have no tumor.

The present disclosure provides a transgenic cell line or host strain comprising the Twa1 gene expression vector as described above. The vector containing Twa1 gene of the present disclosure can be used for transforming a suitable cell line or a host strain. The cell line can be derived from an animal or a plant cell, such as an insect cell, and a mammalian cell. The host strain can be a genetically engineered fungus, Escherichia coli, and yeast. The transgenic cell line or host strain expresses a Twa1 polypeptide or protein.

The present disclosure provides an antibody which is prepared by using any one or more of the following polypeptides or proteins as an antigen, or according to the sequence of any one or several of the following polypeptides or proteins.

1) The amino acid sequence of SEQ ID NO. 2 in the Sequence Listing;

2) A protein which has been subjected to substitution and/or deletion and/or addition of one or several amino acid residues in the amino acid residue sequence of SEQ ID NO. 2 in the Sequence Listing and associated with tumor;

Substitutions and/or deletions and/or additions of one or several amino acid residues refer to substitutions and/or deletions and/or additions of no more than 10 amino acid residues.

3) A polypeptide or protein having 90% or more homology with the amino acid residue sequence of SEQ ID NO. 2 in the Sequence Listing, and associated with tumor.

Wherein, the polypeptide or protein as described above is obtained by artificial synthesis or expression and purification from a transgenic cell line or a host strain as described above. The method for preparing the antibody may be a conventional method such as immunizing an animal, culturing a hybridoma. The antibody may be a polyclonal antibody or a monoclonal antibody, or may be a chimeric antibody, a single chain antibody, a humanized antibody, a Fab fragment, or a product of a Fab expression library. The antibody can be used to detect the abundance of the Twa1 polypeptide or protein of the present disclosure. Since the antibody specifically binds to a Twa1 polypeptide or protein, it can be used for detecting the abundance of Twa1 polypeptide or protein in clinical samples such as human body fluid, blood, cells, tissues. The detection method may be a commonly used method such as immunohistochemistry, immunoblotting, immunofluorescence, ELISA. Depending on the amount of Twa1 polypeptide or protein in the clinical sample, it can be characterized whether the patient providing the sample has a tumor. If a high level of Twa1 polypeptide or protein is detected in a clinical sample, it indicates that the patient is likely to have a tumor. A low level of Twa1 polypeptide or protein in a clinical sample is detected, indicating that the patient is likely to have no tumor.

The present disclosure provides a siRNA (small interfering RNA) for inhibiting or interfering with gene expression, which is designed and synthesized based on a DNA sequence encoding the Twa1 gene as described above. The siRNA is capable of intracellular inhibition or interference with expression of the Twa1 gene. siRNA is usually a 21-nucleotide dsRNA composed of a sense strand nucleotide and an antisense strand nucleotide, which are mainly involved in intracellular RNAi process. The sense strand and the antisense strand are complementary paired. RNAi refers to a phenomenon in which the dsRNA-induced degradation of intracellular mRNA, resulting in the inhibition of target gene expression and loss of biological function. The siRNA of the present disclosure is designed and synthesized based on the sequence of the Twa1 gene, which is capable of inhibiting or interfering with the expression of the Twa1 gene in a specific manner in a cell. By introducing the siRNA into tumor cells, specifically inhibiting or interfering with the expression of the Twa1 gene in tumor cells can efficiently inhibit or interfere with the occurrence and development of tumors. As a major cancer treatment, traditional surgery, radiotherapy and chemotherapy have a strong non-specificity, and they often damage normal cells when killing tumor cells. The siRNA-based gene therapy method has a very high specificity, only interfere with the Twa1 gene described in tumor cells, thus greatly reducing the damage to normal cells and improving the accuracy of gene therapy.

The siRNA provided by the present disclosure is the synthesized dsRNA consisting of a complementary paired sense strand nucleotide and an antisense strand nucleotide. The complementation refers to a relationship in which the nucleotides in the sense strand and the antisense strand can be combined one-to-one. In the structure of double-stranded DNA or some double-stranded RNA molecules, the hydrogen bond between the nucleotides has a fixed number and the distance between the two strands remains unchanged, the nucleotide pairing must follow a certain rule. This is that A (adenine) must be paired with T (thymine) or U (uracil) in RNA, G (guanine) must be paired with C (cytosine), and vice versa.

Preferentially, the sequence of the siRNA is a dsRNA consisting of any one of the following complementary pairs of sense strand nucleotide and an antisense strand nucleotide:

1) sense strand: 5′-GGAGAAGUUUCGAAUGGAATT-3′;
antisense chain: 5′-UUCCAUUCGAAACUUCUCCTT-3′.
2) sense strand: 5′-CAGCGGAGAAGUUUCGAAUTT-3′;
antisense chain: 5′-AUUCGAAACUUCUCCGCUGTT-3′.

The sequences are all composed of 21 nucleotides, the direction of the sequence from left to right is from 5′ end to 3′ end, the positions 1-19 at the 5′ end are ribonucleotides and the positions 20-21 at the 5′ end are deoxyribosenucleotides.

The siRNA provided by the present disclosure is capable of inhibiting or interfering with the expression of the Twa1 gene in a cell. By introducing the siRNA into tumor cells, specifically inhibiting or interfering with the expression of the Twa1 gene in tumor cells efficiently inhibits or interferes with the occurrence and development of tumors. As a major cancer treatment, traditional surgery, radiotherapy and chemotherapy have a strong non-specificity, and they often damage normal cells while killing tumor cells. The siRNA-based gene therapy method is of highly specificity, only interferes with the Twa1 gene described above in tumor cells, greatly reducing the damage to normal cells and improving the accuracy of gene therapy.

The present disclosure provides a shRNA (short hairpin RNA) expression vector for inhibiting or interfering with gene expression. The expression vector is capable of expressing shRNA in cell, and the shRNA is designed and synthesized according to the sequence of the Twa1 gene, which is capable of inhibiting or interfering with the expression of the Twa1 gene in cell. The shRNA expression vector is created by inserting the synthesized shRNA sequence fragment into an existing eukaryotic expression vector, which is capable of expressing the shRNA in a cell and inhibiting or interfering the expression of the Twa1 gene by RNAi in the cell. The shRNA is designed and synthesized based on the sequence of the Twa1 gene, which inhibits or interferes with the expression of the Twa1 gene in a specific manner. By introducing the shRNA expression vector into tumor cell, the shRNA expression vector is capable of expressing the shRNA in cell, specifically inhibiting or interfering with the expression of the Twa1 gene, and effectively inhibiting or interfering with the occurrence and development of tumor. As the main cancer treatment method, traditional surgery, radiotherapy and chemotherapy have a strong non-specificity, and they often have damage to normal cells while killing tumor cells, however the gene therapy method based on the shRNA expression vector described above has a high specificity and only interferes with the Twa1 gene described in tumor cells, which greatly reduces the damage to normal cells and improves the accuracy of gene therapy.

The shRNA provided by the present disclosure is a dsRNA consisting of a complementary paired sense strand nucleotide and an antisense strand nucleotide. The complementation refers to a relationship in which the nucleotides in the sense strand and the antisense strand can be combined in one-to-one. In the structure of double-stranded DNA or some double-stranded RNA molecules, since the hydrogen bond between the nucleotides has a fixed number and the distance between the two strands remains unchanged, the nucleotide pairing must follow a certain rule. This is that A (adenine) must be paired with T (thymine) or U (uracil) in RNA, G (guanine) must be paired with C (cytosine), and vice versa.

Preferentially, the sequence of the shRNA is dsDNA (double-stranded DNA) consisting of any one of the following complementary pairs of sense strand nucleotide and antisense strand nucleotide:

1) sense strand:
5′-GGGAGAAGTTTCGAATGGAATTCAAGAGATTCCATTCGAAAC
TTCTCCCTTTTT-3′;
antisense strand:
5′-AAAAAAGGGAGAAGTTTCGAATGGAATCTCTTGAATTCCATT
CGAAACTTCTCCC-3′;
2) sense strand:
5′-GCAGCGGAGAAGTTTCGAATTTCAAGAGAATTCGAAACTTCT
CCGCTGCTTTTT-3′;
antisense chain:
5′-AAAAAAGCAGCGGAGAAGTTTCGAATTCTCTTGAAATTCGAA
ACTTCTCCGCTGC-3′.

The sense strand sequence consists of 54 deoxyribonucleotides and the antisense strand sequence consists of 55 deoxyribonucleotides, and the direction of the sequence from left to right is from 5′ end to 3′ end.

The shRNA expression vector is capable of inhibiting or interfering with the expression of the Twa1 gene in cell. By introducing the shRNA expression vector into a tumor cell, the shRNA expression vector is capable of expressing the shRNA, specifically inhibiting or interfering with the expression of the Twa1 gene, and effectively inhibiting or interfering with the occurrence and development of tumor. As the major cancer treatment method, traditional surgery, radiotherapy and chemotherapy have strong non-specificity, and often damage normal cells while killing tumor cells. However, the gene therapy method based on the shRNA expression vector described above has a high specificity and only interferes the Twa1 gene in tumor cells, which greatly reduces the damage to normal cells and improves the accuracy of gene therapy.

The present disclosure provides a sgRNA (small guide RNA) expression vector for knocking out a gene. The sgRNA expression vector is capable of expressing sgRNA in cell, and the sgRNA is designed and synthesized according to the sequence of the Twa1 gene. The sgRNA expression vector is capable of knocking out the Twa1 gene in cell. Preferentially, the sgRNA expression vector interacts with a gene editing system in cell, and knocks out the Twa1 gene. Preferentially, the gene editing system is a CRISPR/Cas9 system. The sgRNA expression vector is made by inserting the synthesized sgRNA sequence fragment into a eukaryotic expression vector, and the expression vector is capable of expressing the sgRNA in cell and interacting with the gene editing system in the cell. The Twa1 gene was knocked out completely to inhibit or interfere with the expression of the Twa1 gene. The gene editing system refers to a nucleic acid or protein tool which is capable of editing the Twa1 gene, enabling knock-out, modification, or insertion of a new DNA fragment. The knockout refers to a genetic engineering technique for removing a part of DNA of the Twa1 gene from the DNA sequence of the Twa1 gene, thereby causing loss of function of the Twa1 gene. The modification refers to a genetic engineering technique in which a partial DNA of the Twa1 gene is replaced with another DNA fragment, thereby causing a functional change of the Twa1 gene. The insertion refers to a genetic engineering technique in which another DNA fragment is inserted into the DNA of the Twa1 gene, thereby causing a functional change of the Twa1 gene. The sgRNA is designed and synthesized based on the sequence of the Twa1 gene, and interacts with the gene editing system intracellularly to knock out the Twa1 gene in a specific manner. The sgRNA expression vector is capable of expressing the sgRNA in cell by introducing the sgRNA expression vector into tumor cell, and the sgRNA is intracellularly interacted with a gene editing system to specifically knock out Twa1 gene, which could effectively inhibit or interfere with the occurrence and development of tumors. As the major cancer treatment method, traditional surgery, radiotherapy and chemotherapy have a strong non-specificity, and they often damage normal cells while killing tumor cells. However, the gene therapy method based on the sgRNA expression vector described above has a high specificity and only interferes Twa1 gene in tumor cells, which greatly reduces the damage to normal cells and improves the accuracy of gene therapy.

The sgRNA provided by the present disclosure is a dsRNA consisting of a complementary paired nucleotide sense strand and a nucleotide antisense strand. The complementation refers to a relationship in which the nucleotides in the sense strand and the anti-sense strand can be combined in a one-to-one manner. In the structure of double-stranded DNA or some double-stranded RNA molecules, since the hydrogen bond between the nucleotides has a fixed number and the distance between the two strands remains unchanged, the nucleotide pairing must follow a certain rule. This is that A (adenine) must be paired with T (thymine) or U (uracil) in RNA, G (guanine) must be paired with C (cytosine), and vice versa.

Preferentially, the sequence of the sgRNA is a dsDNA consisting of a complementary pair of sense strand nucleotide and an antisense strand nucleotide:

sense strand: 5′-GAGAGCAGACATGAACCGCC-3′;
antisense strand: 5′-GGCGGTTCATGTCTGCTCTC-3′.

Each sequence is composed of 20 deoxyribonucleotides, and the direction of the sequence from left to right is from 5′ end to 3′end.

The sgRNA expression vector is capable of knocking out the Twa1 gene; Preferentially, the sgRNA expression vector is intracellularly interacting with a gene editing system to knock out the Twa1 gene; Preferentially, the gene editing system is selected from the CRISPR/Cas9 system or other nucleic acid or protein tools which are capable of editing a target gene or genomic locus to knockout, modify, or insert of a new DNA fragment into a particular DNA fragment. The sgRNA expression vector is capable of expressing the sgRNA in cell by introducing the sgRNA expression vector into tumor cell, and the sgRNA is intracellularly interacted with a gene editing system to specifically knock out the Twa1 gene, which will effectively inhibit or interfere with the occurrence and development of tumors. As the major cancer treatment method, traditional surgery, radiotherapy and chemotherapy have a strong non-specificity, and they often damage normal cells while killing tumor cells. However, the gene therapy method based on the sgRNA expression vector described above has a high specificity and only interferes with the Twa1 gene in tumor cells, which greatly reduces the damage to normal cells and improves the accuracy of gene therapy.

The present disclosure provides a protein complex. A protein complex refers to a complex formed by interaction of two or more functionally related proteins. The protein complex of the present disclosure comprises a β-catenin protein and a Twa1 polypeptide or protein, which promotes accumulation of β-catenin protein in the nucleus, thereby promoting tumor cell proliferation. The cell proliferation is an important biological characteristic of cell, and refers to a process in which a cell divides the genetic material into two daughter cells by cell division and forms two daughter cells. The proliferation of normal cells is strictly regulated by the body. Tumor cell proliferation refers to the process in which tumor cells escape from the normal regulation of the body due to genetic mutations and autonomously divide and form daughter cells.

The present disclosure provides a method of inhibiting or interfering with the growth of tumor cells. The method of the present disclosure is to introduce specific nucleic acids into tumor cells to inhibit or interfere with tumor cell growth. The tumor cell growth is a process in which tumor cell escapes from the normal regulation of the body due to a genetic mutation, continuously proliferates to form a tumor, or metastasizes to other organs, and continues to proliferate to form a tumor. As shown in Embodiment 8, in the case of colorectal cancer cells, knockdown of Twa1 in colorectal cancer cells inhibits or interferes with the proliferation and tumorigenic ability of colorectal cancer cells. As shown in Embodiment 11 and Embodiment 12, in the case of gastric cancer cells, overexpression of Twa1 in gastric cancer cells promotes migration and invasion of the cells, and knockout of Twa1 in gastric cancer cells inhibits or interferes with migration and invasion of the cells.

The specific nucleic acid is selected from the group consisting of siRNA as described above, a shRNA expression vector as described above, and an sgRNA expression vector as described above.

The specific nucleic acid inhibits or interferes with the expression of the Twa1 gene as described above or knocks out the Twa1 gene as described above, thereby inhibiting or interfering with tumor cell growth.

The specific nucleic acid inhibits or interferes with the expression of the Twa1 gene as described above or knocks out the Twa1 gene as described above, thereby inhibiting or interfering with the formation of the protein complex as described above, and finally inhibits or interferes with tumor cell growth.

The present disclosure provides a method of inhibiting or interfering with tumor cell proliferation. The method of the present disclosure is to introduce a specific nucleic acid into a tumor cell, thereby inhibiting or interfering with tumor cell proliferation. As shown in Embodiment 8, in the case of colorectal cancer cells, knockdown of Twa1 in colorectal cancer cells inhibits or interferes with the proliferation and tumorigenic ability of colorectal cancer cells.

The specific nucleic acid is selected from the group consisting of siRNA as described above, a shRNA expression vector as described above, and an sgRNA expression vector as described above.

The specific nucleic acid inhibits or interferes with the expression of the Twa1 gene as described above or knocks out the Twa1 gene as described above, thereby inhibiting or interfering with tumor cell proliferation.

The specific nucleic acid inhibits or interferes with the expression of the Twa1 gene as described above or knocks out the Twa1 gene as described above, thereby inhibiting or interfering with the formation of the protein complex as described above, and finally inhibits or interferes with tumor cell proliferation.

The disclosure provides a method of inhibiting or interfering with migration or invasion of tumor cells. The method of the present disclosure is to introduce a specific nucleic acid into a tumor cell, thereby inhibiting or interfering with migration and invasion of the tumor cell. The tumor cell migration refers to a tumor cell invading a lymphatic vessel, a blood vessel or a body cavity from its primary site, and carried by blood flow, lymph flow to another part or organ to continue to grow, forming the same type as the primary tumor. The invasion of the tumor cells refers to invasion or occupation of the malignant tumor from the primary tumor or the secondary tumor to the adjacent host tissue. As shown in Embodiment 11 and Embodiment 12, in the case of gastric cancer cells, overexpression of Twa1 in gastric cancer cells promotes migration and invasion of the cells, and knockout of Twa1 in gastric cancer cells inhibits migration and invasion of the cells.

The specific nucleic acid is selected from the group consisting of siRNA as described above, a shRNA expression vector as described above, and an sgRNA expression vector as described above.

The specific nucleic acid inhibits or interferes with the expression of the Twa1 gene as described above or knocks out the Twa1 gene as described above, thereby inhibiting or interfering with migration and invasion of tumor cells.

The specific nucleic acid inhibits or interferes with the expression of the Twa1 gene as described above or knocks out the Twa1 gene as described above, thereby inhibiting or interfering with the formation of the protein complex as described above, and finally inhibits or interferes with tumor cell migration and invasion.

As shown in Embodiment 13 below, taking gastric cancer cells as an example, knockout of Twa1 in gastric cancer cells causes mesenchymal-epithelial transition in cells, thus Twa1 can promote cell epithelial-mesenchymal transition (EMT), thereby promoting migration and invasion of the tumor cells. The EMT refers to the transformation of epithelial cells to mesenchymal cells, which confers the ability to metastasize and invade cells, including stem cell characteristics, reduced apoptosis and senescence, and enhanced immunosuppression. EMT not only plays a key role in development, but also involved in tissue wound healing, organ fibrosis, and cancer development.

The present disclosure provides the use of any of the methods of inhibiting or interfering with the growth of tumor cells as described above in the preparation or screening of anti-tumor drugs.

The present disclosure provides the use of any of the methods of inhibiting or interfering with tumor cell proliferation as described above in the preparation or screening of anti-tumor drugs.

The present disclosure provides the use of any of the methods of inhibiting or interfering with tumor cell migration or invasion as described above in the preparation or screening of anti-tumor drugs.

The anti-tumor drug may be a cytotoxic drug, a hormonal drug, a biological response modifier, an antibody drug, a cell differentiation inducer, an apoptosis inducer, a neovascularization inhibitor, an epidermal growth factor receptor inhibitor, gene therapy drugs, and tumor vaccines. Preferentially, the anti-tumor drug is selected from the group consisting of an anti-tumor small molecule drug and an anti-tumor small molecule drug composition.

The present disclosure also provides an application of the DNA sequence encoding Twa1 gene, Twa1 polypeptide or protein, siRNA, antibody, shRNA expression vector, sgRNA expression vector, or protein complex in the preparation or screening of antitumor drugs. Preferentially, the application includes preparing the siRNA, shRNA expression vector, or sgRNA expression vector as an antitumor drug, and introducing the drug into tumor cell to specifically inhibit or interfere with Twa1 gene, Twa1 polypeptide or protein, or protein complex, which further inhibits or interferes with tumor cell growth, proliferation, migration, or invasion. Preferentially, the application includes preparing and screening a small molecule drug and small molecule drug compositions that target the protein complex, and introducing the drug or drug composition into tumor cell to specifically inhibit or interfere with the protein complex, which further inhibits or interferes with tumor cell growth, proliferation, migration, or invasion. Preferentially, the application includes preparing and screening a small molecule drug and small molecule drug compositions that target Twa1 gene or Twa1 polypeptide or protein, and introducing the drug or drug composition into tumor cell to specifically inhibit or interfere with Twa1 gene or Twa1 polypeptide or protein, which further inhibits or interferes with tumor cell growth, proliferation, migration, or invasion.

The present disclosure provides an anti-tumor drug, wherein the anti-tumor drug comprises any one or more selected from the group of DNA sequence encoding Twa1 gene, Twa1 polypeptide or protein, siRNA, antibody, shRNA expression vector, sgRNA expression vector, or protein complex, and at least one pharmaceutically acceptable carrier or excipient. The carrier includes the conventional diluents, excipients, fillers, binders, wetting agents, disintegrating agents, absorption enhancers, surfactants, adsorption carriers, or lubricants, and if necessary, flavors or sweeteners agents can be added. The drug of the present disclosure can be made into various forms such as tablets, powders, granules, capsules, oral liquids and injections, and can be prepared according to the conventional methods in the pharmaceutical field. Preferentially, the anti-tumor drug is selected from a cytotoxic drug, a hormonal drug, a biological response modifier, an antibody drug, a cell differentiation inducer, an apoptosis inducer, a neovascularization inhibitor, an epidermal growth factor receptor inhibitor, a gene therapy drug, or a tumor vaccine; more preferentially, the anti-tumor drug is selected from an anti-tumor small molecule drug and an anti-tumor small molecule drug composition.

The present disclosure provides an application of the DNA sequence encoding Twa1 gene, Twa1 polypeptide or protein, siRNA, antibody, shRNA expression vector, sgRNA expression vector, or protein complex in the preparation of a tumor detecting kit. The tumor detection kit refers to a tool for qualitative and quantitative detection of whether a tumor is present or not according to human body fluids, blood, cells, tissues, and other substances reflecting human health status. The kit includes, but is not limited to, a PCR kit, an ELISA kit, a gene chip, or a protein chip.

The present disclosure provides a tumor detecting kit, wherein the kit comprises any one or more selected from the group of DNA sequence encoding Twa1 gene, Twa1 polypeptide or protein, siRNA, antibody, shRNA expression vector, sgRNA expression vector, or protein complex in the application of preparation or screening of antitumor drugs.

The present disclosure provides the application of the DNA sequence encoding Twa1 gene, Twa1 polypeptide or protein, siRNA, antibody, shRNA expression vector, sgRNA expression vector, or protein complex in a drug screening model.

The present disclosure provides a drug screening model, which using any one or more selected from the group of the DNA encoding Twa1 gene, Twa1 polypeptide or protein, siRNA, antibody, shRNA expression vector, sgRNA expression vector, or protein complex as targets to screen the drug that inhibits or interferes with Twa1 expression. The drug screening refers to a process for evaluating biological activity, pharmacological effects, and medicinal value of a substance that may be used as a drug by using an appropriate method, and can be carried out at molecular or cellular level. The drug that inhibits or interferes with Twa1 expression is selected from a cytotoxic drug, a hormonal drug, a biological response modifier, an antibody drug, a cell differentiation inducer, an apoptosis inducer, a neovascularization inhibitor, an epidermal growth factor receptor inhibitor, a gene therapy drug, or a tumor vaccine.

The present disclosure provides a method for diagnosing a tumor, wherein the method is to measure the expression of Twa1 gene or Twa1 polypeptide or protein in human tissues, and up-regulated expression indicates an increase in the probability of cancer.

### BRIEF DESCRIPTION OF THE DRAWINGS

FIG. 1 shows that Twa1 is significantly upregulated in human CRC tissues. (A) Bioinformatics analysis of Twa1 expression in 70 human CRC tissues and 12 nontumor tissues from the Hong CRC microarray data set (GSE9348) available in the Oncomine database. Some genes highly significantly associated with CRC are listed. CXCL1, chemokine (C-X-C motif) ligand 1; DNMT1, DNA (cytosine-5-)-methyltransferase 1; FOXQ1, fork-head box Q1; LGR5, leucine-rich repeat containing G protein-coupled receptor 5. (B) Relative expression of Twa1 mRNA in human CRC tissues and their matched nontumor tissues in the CRC RNA-seq data set obtained from the TCGA database. (C) Relative expression of Twa1 mRNA quantitative RT-PCR (qRT-PCR) analysis of our own clinical samples. Each point represents log 2 transformed Twa1 expression relative to either TBP (TATA binding protein) or small nuclear RNA U6 expression in a single sample. Black horizontal bars show the median±SD. **P<0.0001, Student's t-test. (D) Western analysis of Twa1 protein levels in CRC tissues and their matched nontumor tissues. (E) The densities of Twa1 bands are quantified by Image J software (NIH) and normalized to actin, a loading control. Data are presented as log 2 value of Twa1 (T/N). **P<0.01, Student's t-test.

FIG. 2 shows that Twa1 shRNA (sh-Twa1 and sh-Twa1-2) effectively down-regulates Twa1 polypeptide or protein in HEK-293 cells.

FIG. 3 shows that Twa1 is selectively involved in canonical Wnt signaling. (A) The efficiency of down-regulation of Twa1 protein in HEK-293 cells by lentivirus-based shRNA targeting Twa1 (sh-Twa1) or control shRNA (sh-ctr). (B) Effect of Twa1 depletion on Wnt pathway determined by dual luciferase reporter assays. (C) Effect of Twa1 depletion on Hedgehog pathway. (D) Effect of Twa1 depletion on TNF-α pathway. (E) Effect of Twa1 depletion on Calcium signaling. (F) Effect of Twa1 depletion on TGF-β signaling. (G) Effect of Twa1 depletion on MAPK signaling. (H) Effect of Twa1 depletion on JAK/STAT signaling. (I) Effect of Twa1 depletion on Hippo pathway.

FIG. 4 shows that Twa1 promotes β-catenin nuclear accumulation and target gene expression in response to Wnt signaling. (A) Effect of Twa1 depletion on Wnt-induced luciferase reporter activity. (B) Effect of Twa1 depletion on the Wnt target gene expression. (C) Effect of Twa1 depletion on Wnt-induced luciferase reporter activity. (D) Effect of Twa1 depletion on Wnt target gene expression. (E) Western analysis of endogenous β-catenin and Twa1 from cytosolic and nuclear fractions of HEK-293 cells. (F) Confocal microscopy of β-catenin nuclear localization. (G) Statistical analysis for (F). The intensity of red signals in the nuclei of GFP-positive cells was plotted. (H) Co-IP analysis with the indicated antibodies showing the interaction between endogenous β-catenin and TCF4. (I) Western analysis with the indicated antibodies showing the effect of β-catenin depletion on Twa1 nuclear accumulation upon Wnt3a stimulation.

FIG. 5 shows that knockout of Twa1 suppresses β-catenin nuclear accumulation and Wnt activation. (A) Diagram of the sgRNA target site and the sequence of indels in the Twa1 locus in HEK-293 cells generated by the Cas9/sgRNA system. (B) Agarose gel electrophoresis of the PCR products from the genomic DNA of wild-type (WT) and Twa1 knockout HEK-293 cells (KO-1 and KO-2). (C) Western analysis of endogenous Twa1 in wildtype and Twa1 knockout HEK-293 cells. (D) Effect of Twa1 knockout on Wnt-induced luciferase reporter activity. (E) Effect of Twa1 knockout on Wnt target gene expression. (F) Effect of Twa1 knockout on β-catenin nuclear accumulation by western analysis. (G) Effect of Twa1 knockout on β-catenin nuclear localization by confocal microscopy. (H) Effect of Twa1 knockout on the interaction between endogenous β-catenin and TCF4 by Co-IP experiment.

FIG. 6 shows that Twa1 facilitates Wnt-induced β-catenin nuclear accumulation through its CRA domain. (A) GST pull-down analysis of purified His-β-catenin and wild-type or mutant GST-Twa1 in vitro. LisH, Lis1 homology domain; CTLH, C-terminal to LisH domain; CRA, CT11-RanBPM domain. (B) HEK-293 cells transfected with the indicated constructs were subjected to co-IP and subsequent western analysis. (C) HEK-293 cells infected with sh-Twa1- or sh-ctr-containing lentiviruses were transfected with RNAi-resistant wild-type or mutant human Twa1 constructs, treated with Wnt3a-CM or Ctr-CM, and then processed for immunofluoresence assay. (D) Western blotting analysis. (E) Dual luciferase reporter assays. (F) Wnt target gene expression analysis. (G) HEK-293 cells transfected with wild-type or mutant Twa1 constructs containing NLS sequence were subjected to immunofluoresence analysis. (H) Immunofluoresence analysis. (I) Dual luciferase reporter assays. (J) Wnt target gene expression analysis.

FIG. 7 shows that Twa1 promotes β-catenin nuclear accumulation and Wnt signaling pathway in colorectal cancer cells. (A) DLD1 cells infected with lentiviruses containing sh-Twa1 or sh-ctr were subjected to immunoblotting analysis. (B) Dual luciferase reporter activity. (C) Wnt target gene expression analysis. (D) SW480 cells infected with lentiviruses containing sh-Twa1 or sh-ctr were subjected to immunoblotting. (E) Dual luciferase reporter activity. (F) Wnt-target gene expression analysis.

FIG. 8 shows that Twa1 promotes growth and tumorigenic ability of colorectal cancer cells. (A) MTT assay in DLD1 cells. (B) Colony formation assay in DLD1 cells. (C) MTT assay in SW480 cells. (D) Colony formation assay in SW480 cells. (E) DLD1 cells infected with lentiviruses containing sh-Twa1 or sh-ctr were subcutaneously injected into nude mice. Representative tumors dissected at 28 days post injection are shown (F) Graphs indicate tumor weights

FIG. 9 shows that nuclear Twa1 is associated with CRC cell proliferation and poor prognosis of CRC patients. (A) The expression of nuclear Twa1 and β-catenin in human CRC tissues compared to their corresponding nontumor tissues. Representative images of western blotting show nuclear levels of Twa1 and β-catenin. (B) The densities of bands were quantified by Image J software and normalized to lamin B. Data are presented as log 2 value of Twa1 (T/N). P<0.001, Student's t-test. (C) The correlation between nuclear Twa1 and β-catenin levels was determined by linear regression test (P<0.0001). (D) Kaplan-Meier survival curves for patients with high and low levels of nuclear Twa1 in human CRC tissues. P<0.0001, log rank test.

FIG. 10 shows that Twa1 is significantly upregulated in human gastric cancer tissues. (A) relative expression of Twa1 mRNA in human gastric cancer tissues (T) and nontumor tissues (N) in the gastric cancer RNA-seq data set obtained from the Oncomine database. (B) relative expression of Twa1 mRNA in human gastric cancer tissues (T) and nontumor tissues (N) in the gastric cancer RNA-seq data set obtained from the TCGA database. (C) Kaplan-Meier survival curves for patients with high and low levels of Twa1 in human gastric cancer tissues. The source data is from the GSE57303 dataset of the cancer public database Oncomine. (D) The expression of Twa1 in human gastric cancer tissues compared to their corresponding nontumor tissues. The source data is from clinical tissue samples collected by Zhejiang Cancer Hospital. (E) The expression of Twa1 mRNA in gastric cancer tissues and nontumor tissues by qRT-PCR. The source data is from clinical tissue samples collected by Zhejiang Cancer Hospital. (F) Statistical analysis for (D).

FIG. 11 shows that knockout of Twa1 inhibits the migration and invasion ability of gastric cancer cells. (A) The expression level of Twa1 protein in six types of gastric cancer cell lines and one gastric cancer tissue sample were examined by western blotting. (B) Diagram of the sgRNA target site and the sequence of indels in the Twa1 gene locus in BGC cells generated by the Cas9/sgRNA system. (C) Western analysis of endogenous Twa1 protein level in wild-type and Twa1 knockout BGC cells. (D) The effect of Twa1 knockout on the migration and invasion of BGC cell lines by Transwell assay. (E) Statistical analysis of (D).

FIG. 12 shows that overexpression of Twa1 enhances the migration and invasion ability of gastric cancer cells. (A) Western analysis of endogenous Twa1 in wildtype and Twa1 overexpressed AGS cells and SGC cells. (B) the effect of Twa1 overexpression on the migration and invasion of AGS cells by Transwell assay. (C) the effect of Twa1 overexpression on the migration and invasion of SGC cells by Transwell assay. (D) a statistical diagram of B. (E) a statistical diagram of C.

FIG. 13 shows that knocking out of Twa1 promotes mesenchymal-epithelial transition (MET) in gastric cancer cells. (A) Western analysis of wild-type and Twa1 knockout BGC cells, E-cadherin and Cytokeratin 8 are marker proteins of epithelial cells, while N-cadherin and Vimentin are marker proteins of mesenchymal cells. (B) the cell morphology of wild-type and Twa1 knockout BGC cells.

FIG. 14 shows the up-regulation of Twa1 expression in human bladder cancer, breast cancer, colorectal cancer, lung adenocarcinoma, lung squamous cell carcinoma, pancreatic cancer, prostate cancer, gastric cancer, bronchial adenoma, and thyroid cancer. Ten tumor RNA sequencing data sets were obtained from the cancer public database TCGA.

FIG. 15A-15C show the analysis of the correlation between the expression level of Twa1 and the clinicopathologic features of human pleural cancer, esophageal cancer and renal cancer patients. The results showed that the high expression of Twa1 was related to tumor metastasis (P<0.05). The source data were acquired from the pleural cancer, esophageal cancer and renal cancer pathological information datasets of the cancer public database TCGA.

### DETAILED DESCRIPTION OF THE EMBODIMENTS

The following embodiments are merely illustrative of this disclosure and are not intended to limit the scope of the disclosure. In addition, it should be noted that various modifications and changes may be made to the present disclosure, and the equivalents of the scope of the disclosure.

In order to systematically illustrate the biological function of the Twa1 gene of the present disclosure, colorectal cancer and gastric cancer are exemplified, and the role of the Twa1 gene in the occurrence and development of tumors will be described in detail.

A total of 106 pairs of clinically and surgically removed colorectal cancer tissues and their matched nontumor tissues used in this disclosure were obtained from the Zhejiang Cancer Hospital and the Second Affiliated Hospital of Zhejiang University. Among them, 60 pairs were obtained from Zhejiang Cancer Hospital and 46 pairs were acquired from the Second Affiliated Hospital of Zhejiang University. A total of 80 pairs of clinically surgically removed gastric cancer tissues and their matched nontumor tissues used in the disclosure were obtained from Zhejiang Cancer Hospital. All colorectal cancer tissue samples and gastric cancer tissue samples were obtained and approved by the Ethics Committee of Zhejiang University Medical College. Clinical pathology data were provided by the above hospitals, and at least two professional pathologists independently assessed the clinicopathologic features of the patients.

### Embodiment 1, Twa1 Expression was Significantly Up-Regulated in Human Colorectal Cancer Tissues

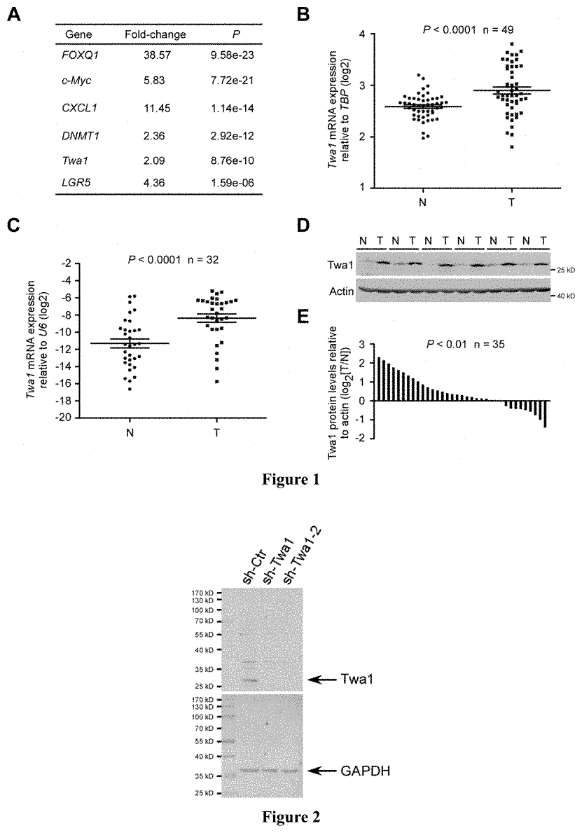

In order to identify the differentially expressed genes in colorectal cancer tissues, the bioinformatics analysis of the Hong colorectal cancer tissue gene expression chip dataset (GSE9348, http://www.oncomine.org) in the cancer public database Oncomine was performed. The data was analyzed using a Affymetrix U133 Plus 2.0 gene chip (Affymetrix, USA), the gene expression levels in 70 colorectal cancer tissues and 12 normal control tissues, and standardized data were obtained using a robust multi-array averaging algorithm. Differentially expressed genes were screened using the R language Limma software package. The linear model and the Empirical Bayes algorithm were used to analyze the data, and the accuracy of the probability P value was evaluated using the FDR (false positive rate) algorithm proposed by Benjiamini and Hochberg. The thresholds of genes considered to be differentially expressed were set as: fold change>2, P<0.01, FDR<0.01. Results in FIG. 1A shows that many genes known to be involved in the development and progression of colorectal cancer are up-regulated in colorectal cancer tissues, namely CXCL1, chemokine (CXC motif) ligand 1; DNMT1, DNA (cytosine-5-)-methyltransferase 1; FOXQ1, fork-head box Q1; LGR5, leucine-rich repeat containing G protein-coupled receptor 5, which indicated that the gene screening method was reasonable and effective. Surprisingly, the results shown in FIG. 1A displayed a previously unknown gene, Twa1, was highly expressed in colorectal cancer tissues. In addition, RNA sequencing data of colorectal cancer tissues in the TCGA (The Cancer Genome Atlas) database (http://cancergenome.nih.gov) was further analyzed. A total of 49 pairs of colorectal cancer tissues and their matched nontumor tissues were included in the database. FIG. 1B showed that the level of Twa1 mRNA was up-regulated in colorectal cancer tissues. Each point in the figure represented the expression level of the Twa1 gene relative to the internal reference gene TBP (TATA binding protein) after log 2 transformation. T indicated tumor tissues and N indicated matched nontumor tissues. The ordinate indicated the expression level of the Twa1 gene relative to the internal reference gene TBP after log 2 transformation. The black horizontal line showed the median±standard deviation. P<0.0001, Student's t test.

To further confirm the expression of Twa1 gene in colorectal cancer tissues, the expression levels of Twa1 mRNA in 32 pairs of matched colorectal cancer tissue samples were detected. The samples were obtained from the Second Affiliated Hospital of Zhejiang University mentioned above. First, the total RNA is extracted from each tissue sample as follows:
(1) Approximately 500 mg of tissue was taken, chopped, placed in a 1.5 ml centrifuge tube, and 1 ml Trizol Reagent (Invitrogene) was added, and homogenization was performed in a tissue disrupter.
(2) 200 μl chloroform was added, shaken vigorously for 15 seconds, and incubated at room temperature for 5 minutes.
(3) Centrifuged at 12000 g for 15 minutes at 4° C.
(4) Carefully transferred the upper aqueous phase to a new 1.5 ml centrifuge tube.
(5) An equal volume of isopropanol was added to the supernatant, and the mixture was allowed to stand at room temperature for 10 minutes.
(6) Centrifuged at 12000 g for 10 minutes at 4° C.
(7) Discarded the supernatant and washed it once with 75% ethanol. Centrifuged at 12000 g for 5 minutes at 4° C.
(8) Discarded the supernatant, dried at room temperature in a fume hood, and dissolved the precipitate in an appropriate amount of nuclease-free DEPC water.
(9) The concentration of total RNA was measured by an ultraviolet spectrophotometer, and 1 μl of the sample was taken for agarose gel electrophoresis, and the extracted total RNA was stored in a −80° C. refrigerator.

Subsequently, the extracted RNA was separately reverse transcribed into cDNA, and the specific steps are as follows:

1) Added the following reagents in a 0.2 ml nuclease-free centrifuge tube:

| Ingredients | Dosage |
|---|---|
| RNA template (5 μg) | 91 |
| Oligod (T) Primer (0.5 μg/μl) | 11 |

Mixed well, incubated at 70° C. for 10 minutes, ice bath for 2 minutes, and then added the following reagents:

| Ingredients | Dosage | |
|---|---|---|
| 5× reaction buffer | 5 | μl |
| dNTP Mix (2.5 mM) | 5 | μl |
| Reverse transcriptase | 1 | μl |
| RNasin | 1 | μl |

| | |
|---|---|
| DEPC ddH2O | added to 25 μl |

3) The mixture was mixed at 42° C. for 1 hour and 72° C. for 10 minutes. The synthesized cDNA was stored at −20° C. Next, based on the human Twa1 gene and human nuclear small RNA U6 gene sequence in the NCBI database, primers were designed according to the primer design principle, and the designed primers were evaluated and screened, and the primers for amplifying the two gene fragments were selected, and the specific primers were selected. The sequence was shown in the table below:

Primers for real-time PCR

| Gene | Primer | sequence (5′→3′) |
|---|---|---|
| Twa1 | upstream | CTGGAAACACTTGATGAACG |
| | downstream | ATCTCTGTGAGGCACTCTCG |
| U6 | upstream | CTCGCTTCGGCAGCACA |
| | downstream | AACGCTTCACGAATTTGCGT |

To ensure the stability and reliability of the experiment, the SYBR GREEN RT-PCR kit from Takara was used in the experiment. Real-time PCR was performed using the cDNA product as a template under optimized conditions. Its reaction system includes:

| Ingredients | Dosage | |
|---|---|---|
| cDNA product | 2 | μl |
| SYBR Premix Ex Taq | 12.5 | μl |
| Upstream primer (10 μM) | 1 | μl |
| Downstream primer (10 μM) | 1 | μl |

| | |
|---|---|
| ddH2O | added to 25 μl |

Amplification conditions: pre-denaturation at 95° C. for 30 seconds, denaturation at 95° C. for 15 seconds, extension at 60° C. for 30 seconds, 40 cycles.

Three independent samples were prepared for each sample, and real-time quantitative PCR was performed according to the above method. The real-time quantitative PCR instrument automatically read the Ct value of each sample according to the standard curve, thereby calculating the copy number in each sample. The quantitative results were averaged, and the copy number of each sample was compared with nuclear small RNA U6, the internal reference gene.

FIG. 1C showed that Twa1 mRNA was significantly up-regulated in colorectal cancer tissues. Each point in the figure represented the expression level of the Twa1 gene relative to the internal reference gene nuclear small RNA (snRNA) U6 after log 2 transformation. T indicated tumor tissues and N indicated the matched nontumor tissues. The ordinate indicated the expression level of the Twa1 gene after log 2 transformation relative to the internal reference gene nuclear small RNA U6. The black horizontal line showed the median±standard deviation. P<0.0001, Student's t test.

1.3 Extracted total protein from colorectal cancer tissues and nontumor tissues, and detected protein expression levels of Twa1 in colorectal cancer tissues. The detailed experimental methods are as follows:

Approximately 500 mg of tissue was taken, washed once with pre-cooled 1×PBS buffer, chopped on ice, then an appropriate amount of RIPA lysis buffer was added and shaken homogenized in a tissue disrupter. The supernatant was collected by centrifugation at 12000 g for 30 minutes at 4° C., and protein quantification was performed using a BCA protein quantification kit. Added 4×SDS loading buffer, mixed and boiled for 10 minutes to denature the protein and store at −20° C.

Next, western blotting was used to detect the expression level of Twa1 protein in various tissues. The detailed experimental steps are as follows:

1.3.1 Polyacrylamide Gel Preparation and Electrophoretic Separation of Proteins

(1) preparing 10% separation gel;

(2) injecting the gel solution and reserving the space required for pouring the concentrated gel at room temperature for 30 minutes to solidify the separated gel;

(3) preparing a 5% concentrated gel;

(4) carefully inserting a clean comb to avoid air bubbles at room temperature for 30 minutes; fixing the gel on the electrophoresis device, adding Tris-glycine running buffer to the tank.

(5) heating the prepared protein sample at 95° C. for 5 minutes;

(6) according to the predetermined procedure, determining the amount for loaded sample according to the comb hole volume and the purpose of the experiment;

(7) turning on the power of the electrophoresis tank, the initial voltage as set at 50 V; after the front edge of the bromophenol blue reached the separation gel, increasing the voltage to 100 V. When the bromophenol blue reached the bottom edge of the separation gel, turned off the power and stopped the electrophoresis.

1.3.2 Transfer Electrophoresis

(1) Membrane Preparation

The PVDF membrane was soaked in methanol for 15 seconds to activate it. Poured 500 ml of pre-cooled transfer buffer into a larger tray, place two sponges, filter papers, and the pre-immersed PVDF membrane into the tray, and fully soak.

(2) Preparation of Gel

Gently pried the glass plate, removed the glass plate, removed the concentrated gel, and cut the glue according to the position of the desired target protein;

(3) Loaded the Transfer Electrophoresis Equipment

Open the clip to keep the black side level. Place a sponge mat on top and use a glass rod to remove the bubbles inside. Then, three layers of filter paper were placed on the mat. Carefully put the gel on the filter paper and adjust the position to align it with the filter paper. Next, put the PVDF membrane on the gel, and then covered the membrane with three layers of filter paper. Finally put another sponge mat on the filter paper.

(4) Electrophoresis

Placed the clip into the transfer slot so that the black side of the clip faced the black side of the groove, and the white side of the clip faced the red side of the groove. Added the transfer buffer to the tank, 100 V and ice bath for 100 minutes.

1.3.3 Blocking

After the transfer electrophoresis, the PVDF membrane was gently rinsed with 1×TBS, shaken slowly in a 5% BSA room at room temperature, and blocked for 1 hour.

1.3.4 Antibody Incubation

(1) Primary Antibody Incubation

The primary antibodies were diluted with blocking solution (5% BSA) according to the antibody instructions, incubated for one hour at room temperature, and then overnight at 4° C. On the next day, incubated at room temperature for another hour.

(2) Washed Membrane with 1×TBST

Rapid washed the membrane on a shaker, 10 minutes×3 times;

(3) Secondary Antibody Incubation

The antibody dilution (5% BSA) was diluted at 1:5000 with a fluorescent secondary antibody, shaken slowly at room temperature, and incubated for 1 hour in the dark.

(4) Washed Membrane with 1×TBST

Rapid washed the membrane on a shaker, 10 minutes×3 times;

1.3.5 Scanning

Used the Odyssey fluorescence scanning system, selected the appropriate scan intensity and scanned the fluorescence of the target protein.

Twa1 antibody was purchased from Wuhan Sanying Company, China, GAPDH antibody was purchased from American Sigma Company, and fluorescent secondary antibody was purchased from American LICOR Company. FIG. 1D showed that Twa1 protein was up-regulated in colorectal cancer tissues. N represented nontumor tissues. and T represented colorectal cancer tissues. Actin is an internal reference protein, indicating the total protein amount of N and T used for pairwise comparison. If the intensities of actin bands among each sample were consistent, the total protein amount in N and T would be consistent. The internal reference protein is generally referred to a protein encoded by the housekeeping gene. Their expression in each tissue and cell is relatively constant, and it is often used as a reference when comparing changes in the expression level of a protein. The western blotting experiment was used to compare the relative expression of the target protein under different conditions or in different tissue cells. If the same amount of protein is loaded, and the basis of comparison is obtained. FIG. 1E was a quantitative statistical plot of FIG. 1D, and the intensities of the Twa1 band were quantified and normalized by Image J software (NIH, National Institutes of Health). The ordinate indicated the expression level of the Twa1 protein relative to the internal reference actin protein after log 2 transformation. P<0.01, Student's t test.

The above results indicate that Twa1 is highly expressed in colorectal cancer tissues and may be associated with the development and progression of colorectal cancer.

Embodiment 2, a vector for the inhibition of Twa1 gene expression and a lentivirus were prepared. Detailed steps are as follows:

(1) According to the human Twa1 mRNA sequence in NCBI, two different shRNA targets were designed for this sequence, and the corresponding sense strand and antisense strand nucleotides (Shanghai Shenggong) were synthesized. Two shRNAs were designed and synthesized. shRNA was a dsDNA consisting of a set of complementary pairs of sense strand nucleotide and an antisense strand nucleotide:

1) sense strand:
5′-GTTCCATTCGAAACTTCTCCTTCAAGAGAGGAGAAGTTTCGA
ATGGAACTTTTT-3′;
antisense strand:
5′-AAAAAAGTTCCATTCGAAACTTCTCCTCTCTTGAAGGAGAA
GTTTCGAATGGAAC-3′;
2) sense strand:
5′-GATTCGAAACTTCTCCGCTGTTCAAGAGACAGCGGAGAAGTT
TCGAATCTTTTT-3′;
antisense strand:
5′-AAAAAAGATTCGAAACTTCTCCGCTGTCTCTTGAACAGCGGA
GAAGTTTCGAATC-3′.

The above shRNA sequences were separately constructed into the lentiviral vector pGLV-U6/GFP (Genepharma, Shanghai). The constructed shRNA expression vectors were named sh-Twa1 and sh-Twa1-2.

(2) HEK-293 cells were transfected with Lipofectamine 2000 (Invitrogene) according to manufactural instructions. The sh-Twa1, sh-Twa1-2 and sh-ctr control vectors and the viral packaging plasmid (Genepharma, Shanghai) were co-transfected into HEK-293T cells (ATCC cell bank) for 48 hours, and then the medium containing the virus was collected, filtered, and mixed with 4 μg/ml polybrene (Shanghai Shenggong) to treat cells.

(3) HEK-293 cells (Shanghai University of Chinese Academy of Sciences) were infected with the lentivirus for 48 hours, and the total protein from infected cells was extracted for immunoblotting to detect the expression of Twa1 protein.

As shown in FIG. 2, the lentivirus containing the shRNA of Twa1 was able to significantly down-regulate the protein level of Twa1 in HEK-293 cells. GAPDH (glyceraldehyde-3-phosphate dehydrogenase) was an internal reference protein.

### Embodiment 3, Down-Regulation of Twa1 Significantly Inhibited the Activity of the Wnt Signaling Pathway Reporter Gene

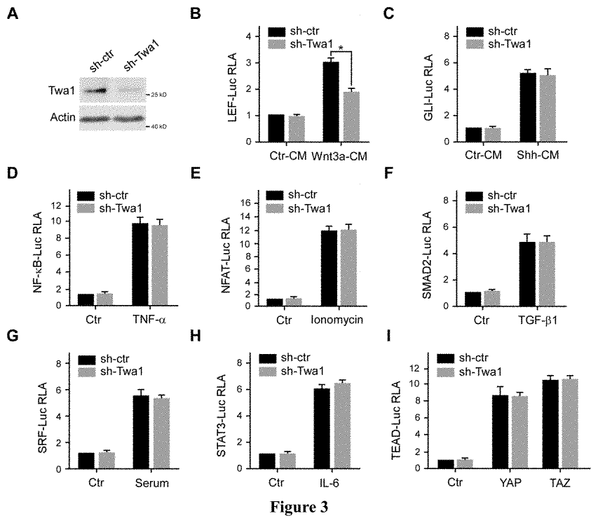

Aberrant cell signaling pathway is an important cause of colorectal cancer. To explore whether Twa1 participates in the regulation of signaling pathways involved in the pathogenesis of colorectal cancer, a series of dual luciferase reporter gene experiments reflecting the activity of signaling pathways were carried out (Involved in Wnt, TGF-β, Hedgehog, Hippo, NF-κB, MAPK, JAK/STAT, and calcium signaling). First, HEK-293 cells were infected with lentivirus containing sh-Twa1 or sh-ctr, and immunoblotting results were shown in FIG. 3A. The lentivirus containing sh-Twa1 significantly down-regulated the Twa1 protein level in HEK-293 cells. The reporter gene plasmid of the above signal pathway and the Renilla luciferase (internal reference) plasmid were transfected, and the relevant signal pathways were activated by the corresponding pathway activators. The activities of these two types of luciferase were detected by a microplate reader. The experiment was carried out using dual luciferase reporter gene kit (Promega). The detailed experimental methods are as follows.

(1) HEK-293 cells were infected with the lentivirus containing sh-Twa1 or sh-ctr for 48 hours.

(2) Transfected the reporter plasmids and the internal reference pRL-TK (Promega) into cells in a 12-well plate.

(3) After 24 hours of transfection, cells were treated with signaling activators according to experimental requirements.

(4) 200 μl of PLB cell lysate was added to each well of a 12-well plate to lyse the cells.

(5) Shaken the 12-well plate on a shaker for 20 minutes at room temperature and transferred the cell lysate into a 1.5 ml centrifuge tube.

(6) Centrifuged at 12000 g for 30 seconds, pelleted the cells, and transferred the supernatant in a new centrifuge tube.

(7) Transferred 20 μl supernatant into a small well of a 96-well microtiter plate and set the program for detection.

(8) Added 20 μl LARII to detect the fluorescence activity of Firefly. Subsequently, 20 μl Stop & Glo Reagent was added to the well to detect Renilla fluorescence activity. The ratio of the Firefly fluorescence activity to the Renilla fluorescence activity value was the final result. Repeated 3 times for each experiment. *P<0.05, Student's t test.

FIG. 3B showed that down-regulation of Twa1 specifically inhibited the activity of the canonical Wnt signaling pathway reporter gene. The ordinate in the figure indicated the value of the reporter gene activity. The panels C, D, E, F, G, H, and I in FIG. 3 showed that down-regulation of Twa1 had no significant effect on these pathways. The ordinate in the figure indicated the value of the reporter gene activity. GLI, glioma-associated genes (Hedgehog signaling pathway); IL-6, interleukin-6; LEF, lymphoid enhancer binding factor (Wnt/β-catenin signaling pathway); Luc, luciferase; NF-κB, nuclear factor-κB (TNF-α signaling pathway); NFAT, T cell nuclear activating factor (calcium signaling pathway); RLA, relative luciferase activity (firefly/renilla luciferase); Shh-CM, Shh conditioned medium; Smad2 SMAD family member 2 (TGF-β signaling pathway); SRF, serum response factor (MAPK signaling pathway); STAT3, signal transduction and transcriptional activator 3 (JAK/STAT signaling pathway); TAZ, with PDZ binding motif Transcriptional coactivator; TEAD, TEA domain (Hippo signaling pathway). TGF-β1, transforming growth factor β1; TNF-α, tumor necrosis factor-α; Wnt3a-CM, Wnt3a conditioned medium; YAP, Yes related protein.

These data suggest that Twa1 is required for the canonical Wnt signaling pathway.

### Embodiment 4, Twa1 Promoted β-Catenin Nuclear Accumulation and Wnt Target Gene Expression

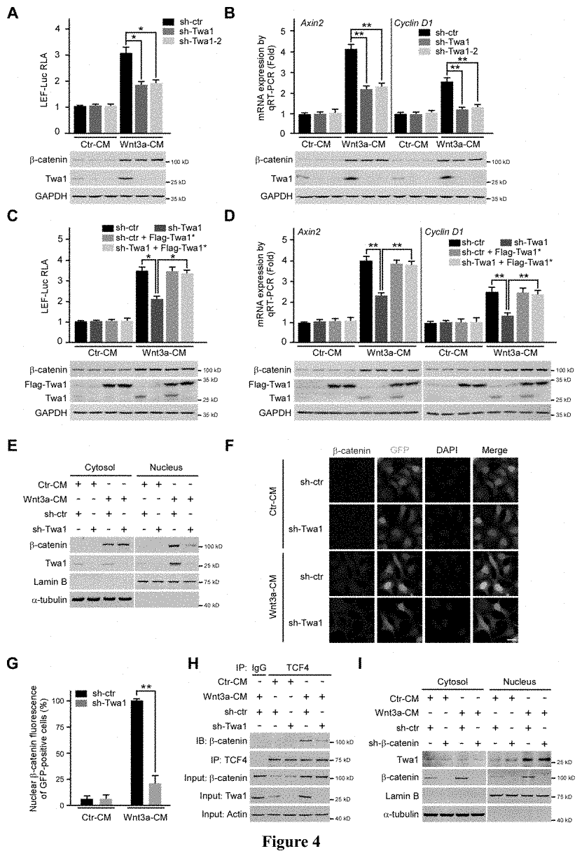

4.1 As shown in FIG. 4, knockdown of Twa1 by using two different shRNA significantly inhibited Wnt reporter activity. The canonical Wnt signaling pathway plays a biological role by regulating the expression of downstream target genes, and thus the inventors examined the effect of knockdown of Twa1 on the expression of Wnt target genes Axin2 and Cyclin D1. HEK-293 cells depleted with Twa1 or not, were treated with Wnt3a conditioned medium or control medium for 6 hours, and total cellular RNA was extracted and reverse transcribed into cDNA. As shown in FIG. 4B, real-time PCR experiments displayed that knockdown of Twa1 decreased expression of Wnt target genes Axin2 and Cyclin D1. The ordinate of the graph indicates the relative expression levels of Axin2 and Cyclin D1. To exclude the off-target effect of Twa1 shRNA, a Twa1 expression plasmid resistant to Twa1 shRNA was transfected. The dual luciferase reporter gene assay (FIG. 4C) and real-time PCR (FIG. 4D) showed that ectopic expression of Twa1 resistant to Twa1 shRNA could successfully rescue the phenotype caused by Twa1 knockdown, which confirmed the effectiveness and specificity of Twa1 shRNA and excluded the off-target effects. Quantitative data were expressed as mean±standard error (at least three independent experiments). *P<0.05, **P<0.01, Student's t test. The method for extracting total RNA from cells and the primer sequences used in the experiment are as follows:

Added 1 ml Trizol to 35 mm culture dish, lysed the adherent cells on the bottom surface, pipetted the lysate into the RNase-free centrifuge tube, added 0.2 ml pre-cooled chloroform, mixed well, shaken vigorously for 15 seconds, and let the tube stand at room temperature 2-3 minutes, cleared and stratified; centrifuged at 12000 g for 15 minutes at 4° C.; drew the upper RNA-containing aqueous phase to a new 1.5 ml tube, added 0.5 ml of pre-cooled isopropanol, mixed and let the tube stand for 20 minutes; 4° C., 12000 g for 10 minutes; discarded the supernatant, resuspended the RNA pellet in 1 ml 70% ethanol, and centrifuged at 7500 g for 5 minutes at 4° C.; dried at room temperature until the ethanol is evaporated, added 20 μl of RNase-free H2O; took 3 μl for quantification, and the RNAs were stored at −80° C.

Primers for real-time PCR

| Gene | primer | sequence (5′→3′) |
|---|---|---|
| Axin2 | upstream | CTGGCTCCAGAAGATCACAAAG |
| | downstream | ATCTCCTCAAACACCGCTCCA |
| Cyclin D1 | upstream | AGCTCCTGTGCTGCGAAGTGGAA |
| | downstream | AGTGTTCAATGAAATCGTGCGGG |
| GAPDH | upstream | GCACCACCAACTGCTTA |
| | downstream | AGTAGAGGCAGGGATGAT |

4.2 To elucidate the molecular mechanism by which Twa1 regulates the canonical Wnt signaling pathway, the level of β-catenin, a key molecule of this pathway, was examined. HEK-293 cells were first infected with lentivirus containing sh-Twa1 or sh-ctr for 48 hours. The lentivirus-infected HEK-293 cells were subsequently treated with Wnt3a conditioned medium and control medium for 6 hours. Western blotting experiments (FIGS. 4B and 4D) showed that down-regulation of Twa1 had no significant effect on the overall level of β-catenin. The extraction method of total intracellular protein is as follows:

(1) placing cells on a six-well culture plate at a density of 1×106, and 2 ml DMEM medium containing 10% fetal calf serum was added to each well.

(2) adding 100 μl RIPA buffer (containing 0.1 M DTT, 1 mM Na3VO4, 1 mM NaF, 1 mM PMSF) to each well, scraped the cells with a scraper, pipetted the cell lysate into a 1.5 ml centrifuge tube, and incubated at 4° C. for 35 minutes.

(3) centrifuging at 14000 g for 5 minutes, aspirating the supernatant, adding 4× loading buffer, and boiling for 5 minutes.

(4) after boiling, immediately putting on ice and stored at −20° C.

4.3 Since β-catenin translocates from the cytoplasm to the nucleus to regulate Wnt target gene expression, the inventors tested whether down-regulation of Twa1 affected the protein level of β-catenin in the nucleus. The cytoplasmic and nuclear components of the cells were isolated using a Nuclear Extract kit (Active Motif, USA) to detect the expression of β-catenin in the nucleus. The detailed experimental methods are as follows:

(1) after the medium was aspirated, washing cells twice with 1×PBS, scraping off with a scraper collected into 2 ml centrifuge tubes.

(2) adding 0.5 ml Hypotonic Buffer to each tube and placing on ice for 15 minutes while gently mixing.

(3) adding Detergent, mixing and vortexing for 20 seconds, centrifuged at 12000 g for 1 minute, taking the supernatant and precipitate separately, and centrifuging the supernatant at 14000 g for 5 minutes at 4° C., then taking the supernatant for cytoplasmic extraction.

(4) washing the precipitate by adding 1 ml pre-cooled hypotonic buffer, centrifuging at 14,000 g for 1 minute at 4° C., and removing the supernatant.

(5) adding 0.2 ml cell lysis buffer. The precipitates were resuspended by shaking, shaken gently on a 4° C. shaker for 20 minutes, centrifuged at 14,000 g for 10 minutes at 4° C., and the supernatant was taken as a nuclear extract.

Adding 4×SDS loading buffer to the prepared cytoplasm and nuclear fraction, mixed and boiled for 10 minutes to denature the protein and stored at −20° C.

Western blotting experiments showed that down-regulation of Twa1 significantly reduced the protein level of β-catenin in the nucleus (FIG. 4E). Lamin B and α-tubulin were used as the internal reference proteins for the nucleus and cytoplasm, respectively.

4.4 Immunofluorescence experiments showed that knockdown of Twa1 inhibited the localization of β-catenin in the nucleus. HEK-293 cells were infected with lentivirus containing sh-Twa1 or sh-ctr for 48 hours. The lentivirus-infected HEK-293 cells were subsequently treated with Wnt3a conditioned medium and control medium for 6 hours, followed by immunofluorescence experiments. FIG. 4F showed that when the Wnt signal was inactivated, β-catenin was mainly localized on the cell membrane, and its levels in the cytoplasm and nucleus were lower. Activation of Wnt signaling by Wnt3a conditioned medium significantly enhanced the expression and distribution of β-catenin in the cytoplasm and nucleus of the control cells. However, down-regulation of Twa1 inhibited the distribution of β-catenin in the nucleus but had no significant effect on its expression and localization in the cytoplasm, suggesting that Twa1 is critical for β-catenin nuclear accumulation. The green signal in the figure represented the cells infected with lentivirus, the red signal characterized the β-catenin protein, and the DAPI indicated DNA (blue). Bars, 10 μm. The experimental procedures are as follows:

(1) The cells were seeded on the coverslips placed in a 6-well plate. After the experiment as mentioned above, the cells were fixed with 4% formaldehyde for 10 minutes.

(2) Cells were gently rinsed with 1×PBS three times for 5 minutes each time, blocked with 10% FCS in PBS for 20 minutes at room temperature, and the corresponding primary antibody containing 0.1% saponin was incubated at room temperature (prepared with 10% FCS/PBS) for 2 hours or 4° C. overnight.

(3) Cells were gently rinsed with 1×PBS three times for 5 minutes each time, and the corresponding fluorescent secondary antibody containing 0.1% Saponin and DAPI was incubated for 1 hour at room temperature in the dark.

(4) Cells were gently rinsed with 1×PBS three times for 5 minutes each time. The coverslips were mounted on glass slides with the mounting medium and sealed with the nail polish. The sealed slides were stored at 4° C.

(5) Image acquisition was performed using a Zeiss LSM510 confocal microscope. The image acquisition was performed using a 63× objective with an NA value of 1.4 in a multi-channel mode.

4.5 Co-immunoprecipitation experiments showed that down-regulation of Twa1 inhibited the interaction of β-catenin with the transcription factor TCF4. In the nucleus, β-catenin activates downstream target gene expression by binding to the transcription factor TCF4. Since down-regulation of Twa1 decreased the nuclear level of β-catenin, it is speculated that a decrease in the expression of Twa1 will reduce the amount of β-catenin bound to TCF4. HEK-293 cells infected with a lentivirus containing sh-Twa1 or sh-ctr were treated with Wnt3a conditioned medium or control medium for 6 hours. Subsequently, each group of cell lysates was extracted, and an anti-β-catenin antibody or a control IgG antibody was used for co-immunoprecipitation experiments, and the level of TCF4 was detected by immunoblotting. The results of co-immunoprecipitation experiments in FIG. 4 showed that the down-regulation of Twa1 indeed resulted in a decrease in the amount of β-catenin bound to TCF4, further confirming that Twa1 is involved in the regulation of β-catenin nuclear accumulation and affects the canonical Wnt signaling pathway. The experimental process of co-immunoprecipitation is as follows:

4.5.1 Protein Extraction, Binding to Antibodies

Washed and dried the cell scraper in advance, wrapped it in plastic wrap and placed on ice. The 1.5 ml centrifuge tube was placed on ice for pre-cooling.

(1) washing the cells twice with pre-cooled 1×PBS, and aspirating the 1×PBS.

(2) 300 μl IP lysis buffer (50 mM Hepes [pH 7.4], 150 mM NaCl, 1% Nonidet P-40, 10% glycerol, 10 mM EGTA, 1.5 mM MgCl2, 1 mM PMSF, and protease inhibitor cocktail) to a 100 mm diameter cell culture dish. Incubated for 2 minutes, scraped the cells and transferred to a 1.5 ml centrifuge tube.

(3) After vortexing and mixing, rotated at 4° C. for 30 minutes.

(4) 15000 g, centrifuged at 4° C. for 20 minutes.

(5) Pipetted the supernatant to a pre-cooled centrifuge tube, and take 40 μl the supernatant to another centrifuge tube for use as an input.

(6) Added 1 μg the antibody to the extracted protein lysate and incubate overnight at 4° C.

4.5.2 Antibody and Protein Complex Binding with Protein A/G Beads (Santa Cruz)

(1) Removed the appropriate amount of Protein A/G beads into a pre-cooled 1.5 ml centrifuge tube.

(2) The beads were washed once with the pre-cooled wash buffer (50 mM Hepes [pH 7.4], 150 mM NaCl, 1% NP-40, 10% glycerol, 10 mM EGTA, 1.5 mM MgCl2). Centrifuged at 4° C., 3000 rpm, 2 minutes, tried to remove the liquid.

(3) The antibody and protein complex were transferred into the washed Protein A/G beads.

(4) Incubated for 2 hours at 4° C.

(5) The beads were washed 4 times with IP lysis buffer containing 0.5% NP-40, and finally washed once with pre-cooled 1×PBS.

Note: when co-immunoprecipitation experiments were carried out with Flag antibody-conjugated beads, the beads were directly added to the protein lysate and incubated at 4° C. for 4 hours and then washed.

4.5.3 Immunoprecipitates for Western Blotting

(1) Added an appropriate amount of 2× loading buffer.

(2) The sample was boiled for 5 minutes and immediately placed on ice.

(3) Centrifuged, took 20 μl the supernatant for western blot to detect the precipitated protein in the antibody and protein complex.

### Embodiment 5, Knockdown of Twa1 Inhibited β-Catenin Nuclear Accumulation and Wnt Signaling Pathway

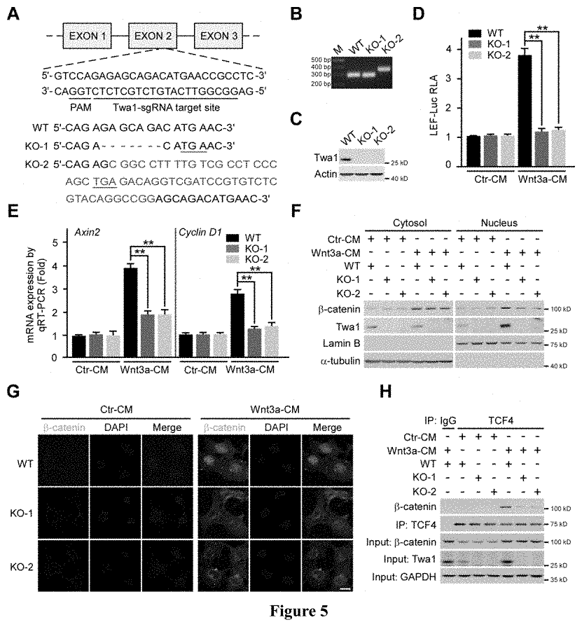

To further verify that Twa1 promotes β-catenin nuclear accumulation and Wnt signaling, Twa1 gene was knocked out in HEK-293 cells using CRISPR/Cas9 technology, and the effect of Twa1 gene deletion on β-catenin nuclear accumulation and Wnt signaling pathway was examined. FIG. 5A showed the sequencing results of the Twa1 gene in normal cells, and the sequencing results of the Twa1 gene edited by the CRISPR/Cas9 system in two Twa1 knockout cells. In Twa1-KO-1 and Twa1-KO-2 cells, the protein translation of the edited Twa1 gene was prematurely terminated, thus Twa1 protein was successfully knocked out. FIG. 5B showed the size of the PCR products amplified from Twa1 gene in normal cells, and in Twa1-KO-1 and Twa1-KO-2 cells, indicating that the Twa1 gene editing did occur as shown in FIG. 5A. The results of the western blotting experiments were shown in FIG. 5C. Knockout of Twa1 gene completely abolished Twa1 protein expression. As shown in FIG. 5D-H, knockout of the Twa1 gene significantly inhibited β-catenin nuclear accumulation, Wnt reporter activity, Wnt target gene expression, and the interaction of β-catenin with TCF4. In FIG. 5G, green signal represented β-catenin and DAPI represented DNA (blue). Scale bars, 10 μm. Quantitative data were expressed as mean±standard error (at least three independent experiments). N.S., not significant. **P<0.01, Student's t test. These results confirm that Twa1 promotes β-catenin nuclear accumulation and Wnt signaling pathway. The detailed methods for establishing Twa1 knockout cells are as follows:

(1) A pair of sgRNA sequences (5′-GAGAGCAGACATGAACCGCC-3′ and 5′-GGCGGTTCATGTCTGCTCTC-3′) were synthesized and inserted into the modified pEP330X vector system.

(2) The vector was transfected into HEK-293 cells, and the cells were treated with 1 μg/ml of puromycin for 24 hours. The cells were then seeded in 96-well plates to form clones.

(3) Extraction of genomic DNA from a single colony. PCR amplification was carried out using primers (5′-ATTCTCCGGCTCACAGCTC-3′ and 5′-GCTACAGCACTCCTTATGTGTT-3′), and positive clones were verified by genomic DNA sequencing and immunoblotting experiments.

### Embodiment 6, Twa1 Promoted Nuclear Accumulation in β-Catenin by Binding to β-Catenin

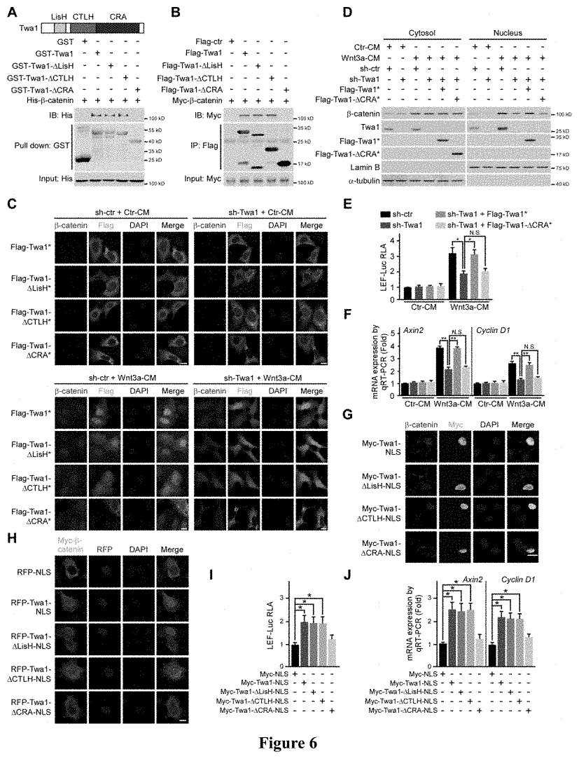

6.1 To demonstrate the molecular mechanism by which Twa1 regulates nuclear accumulation in the β-catenin, the interaction between Twa1 and β-catenin was examined. GST-tagged Twa1 or His-tagged β-catenin fusion protein was expressed and purified from E. coli for GST pull-down assays in vitro., FIG. 6A showed that Twa1 and β-catenin could bind directly. Co-immunoprecipitation experiments further confirmed their interaction (FIG. 6B). To identify the binding region where Twa1 interacts with β-catenin, the domain structure of Twa1 was analyzed by bioinformatics methods. Twa1 has three domains: an N-terminal LisH domain, a central CTLH domain, and a C-terminal CRA domain. Twa1 mutants deleted of each domain were constructed: LisH mutants lacking the LisH domain (Twa1-ΔLisH), Twa1 mutants lacking the CTLH domain (Twa1-ΔCTLH), and mutants lacking the CRA domain (Twa1-ΔCRA), and the mutant proteins were expressed and purified. The GST pull-down experiment showed that Twa1-ΔCRA cannot bind to β-catenin, while the deletion of LisH domain or CTLH domain had no significant effect (FIG. 6), suggesting that the CRA domain is important to Twa1 and β-catenin. The wild-type Twa1 and the mutants were expressed in cells for co-immunoprecipitation experiments. FIG. 6B showed that Twa1 lacking the CRA domain failed to interact with β-catenin, and Twa1-ΔLisH or Twa1-ΔCTLH was still capable of binding to β-catenin. These results indicate that Twa1 interacts with β-catenin via the CRA domain. The GST or His pull-down experiments are as follows:

6.1.1 GST Fusion Protein Purification

6.1.1.1 Plasmid Transformation Using BL21 Strain

(1) 100 μl competent cell (Vazyme) stored in a −80° C. refrigerator was thawed on ice.

(2) Added the plasmid to the competent cell and shaken gently, and placed on ice for 30 minutes.

(3) 42° C. water bath for 90 seconds, quickly placed on ice for 2 minutes.

(4) Added 800 μl pre-warmed LB liquid medium (without antibiotics) to the tube, mixed and incubated at 37° C. for 45 minutes.

(5) Added 100 μl cell on a culture plate, and placed the plate at 37° C. overnight.

6.1.1.2 Induction of Fusion Protein Expression

(1) The bacteria clones were picked up and placed into a tube with 5 ml LB medium containing antibiotic.

(2) 37° C., 200 rpm, shaken overnight (12 hours).

(3) Added 5 ml bacterial solution to another bottle with 200 ml LB medium containing antibiotics. 200 rpm, shaken for 1-2.5 hours.

(4) When the OD600 value of the bacterial solution reached 0.4-0.6, 1 mM IPTG was added to induce protein expression at 16° C. for 16 hours.

(5) Centrifuged at 4° C., 5000 rpm for 5 minutes, which allowing the bacteria to precipitate (can be stored at −20° C.).

6.1.1.3 Purified Protein

(1) 60 ml of pre-cooled PBS (containing 1 mM PMSF and 2 mM EDTA) was added to the bacterial. Sonicated the cells on ice with 50% power. One second, stopped for one second, over 90 times. Repeated 3 times until the liquid became clear.

(2) Centrifugation, 4° C., 5000 g, 30 minutes. Transferred the supernatant to another clean 50 ml centrifuge tube.

(3) Added 500 μl pre-washed GST-beads (the beads are washed 3 times with pre-cooled PBS to remove the alcohol from the beads). Incubated for 2 hours on a shaker at 4° C.

(4) Centrifuged at 3000 rpm for 1 minute and discarded the supernatant.

(5) Added 5 ml pre-cooled PBS (containing EDTA, PMSF) and wash 5 times for 5 minutes each time.

(6) Added 1 ml GSH (glutathione) solution and the tube was rotated at 4° C. for 5 minutes. Centrifuged and transferred the supernatant to a new tube. Eluted 4-5 times and collected the fractions eluted each time.

(7) Aspirated the eluted fraction into a dialysis bag for dialysis. First, immersed the dialysis bag in distilled water, then sealed the dialysis bag with a clip in one end, then rinsed the bag 2-3 times with distilled water. Added the protein solution to the bag and sealed the other end with a clip. The dialysis bag containing the protein eluate was shaken overnight in GSH-free dialysate (containing EDTA 2 mM). On the next day, transferred the bag to a fresh dialysate (without EDTA) at 4° C. for 4 hours.

(8) Concentrated the protein at 4° C. The dialysis bag was immersed in pre-cooled sucrose and fresh sucrose was exchanged every 2 hours for 3 times.

6.1.2 GST Pull-Down Experiment

(1) An equimolar amount of GST-Twa1 protein or a mutant protein was added to the purified His-β-catenin protein, respectively.

(2) Incubated overnight at 4° C.

(3) Washed GST-beads three times with PBS. Added 30 μl beads to each tube. Incubated for 2 hours at 4° C.

(4) Washed the beads 5 times with PBS, 1 ml each time, and centrifuged at 2500 rpm for 1 minute. Tried to remove all the supernatant. Added 50 μl/tube of 2× loading buffer to the beads and boiled for 5 minutes.

(5) Western blotting to detect the targeted protein.

6.2 To explore whether Twa1 regulates β-catenin nuclear accumulation through the CRA domain, wild-type Twa1 and its mutants were expressed in Twa1-depleted HEK-293 cells. Immunofluorescence experiments showed that in the absence of Wnt signaling, Twa1 and its domain-deleted mutants were mainly localized in the cytoplasm, and β-catenin was mainly distributed on the cell membrane and cytoplasm (FIG. 6C). Upon Wnt signaling, Twa1, Twa1-ΔLisH, and Twa1-ΔCTLH entered the nucleus, and β-catenin also translocated into the nucleus, indicating that the Twa1 mutant lacking the LisH domain and CTLH domain could successfully rescue the phenotype induced by Twa1 knockdown. However, in cells transfected with Twa1-ΔCRA, Twa1-ΔCRA itself did not translocate into the nucleus, and β-catenin also did not accumulate in the nucleus, suggesting that the CRA domain is critical for Twa1 to regulate β-catenin nuclear accumulation.

6.3 To examine the effect of Twa1 mutants on Wnt target gene expression. Western blotting showed that the wild-type Twa1 can rescue the decrease of the β-catenin nuclear level caused by the down-regulation of Twa1, while the Twa1-ΔCRA could not reverse the abnormal phenotype. The dual luciferase reporter gene assay in FIG. 6E and the real-time PCR assay in FIG. 6F show that wild-type Twa1 can rescue the reduction of Wnt gene activity and the expression of target genes Axin2 and CyclinD1 caused by down-regulation of Twa1. In contrast, Twa1-ΔCRA could not reverse the abnormal phenotype, further confirming that Twa1 regulates Wnt signaling through the CRA domain.

6.4 Wnt signaling promotes β-catenin nuclear accumulation by increasing the nuclear level of Twa1 nucleus. To investigate whether nuclear Twa1 is sufficient to enhance β-catenin nuclear localization by the CRA domain even in the absence of Wnt signaling, the inventors fused a nuclear localization sequence (NLS) at the C-terminus of Twa1 and its mutants to force their expression in the nucleus. Without Wnt stimulation, either exogenous GFP-β-catenin or endogenous β-catenin was localized in the cytosol (FIGS. 6G and H). However, GFP-β-catenin or endogenous β-catenin translocated into the nucleus in cells with NLS-Twa1 expression, suggesting that promoting Twa1 nuclear accumulation is sufficient to enhance β-catenin nuclear localization. Deletion of the LisH domain or CTLH domain of NLS-Twa1 had no significant effect on the function of Twa1, and β-catenin was still localized in the nucleus. However, the NLS-Twa1 mutant lacking the CRA domain, even if it was capable to localize in the nucleus, failed to promote β-catenin nuclear accumulation. In FIG. 6G, red signal represented β-catenin, green signal represented NLS-Twa1 or its mutants, and DAPI indicated DNA (blue). Scale bar, 10 μm. In FIG. 6H, green represented GFP-β-catenin, red represented NLS-RFP-Twa1 or its mutants, and DAPI indicated DNA (blue). Scale bar, 10 μm. Dual luciferase reporter gene assay in FIG. 6I and the real-time PCR results in FIG. 6J further confirmed that NLS-Twa1 promoted Wnt target gene expression by the CRA domain. Taken together, these data indicate that Twa1 bind to β-catenin and promotes β-catenin nuclear accumulation via the CRA domain.

### Embodiment 7, Twa1 Promoted β-Catenin Nuclear Accumulation and Wnt Signaling Pathway in Colorectal Cancer Cells

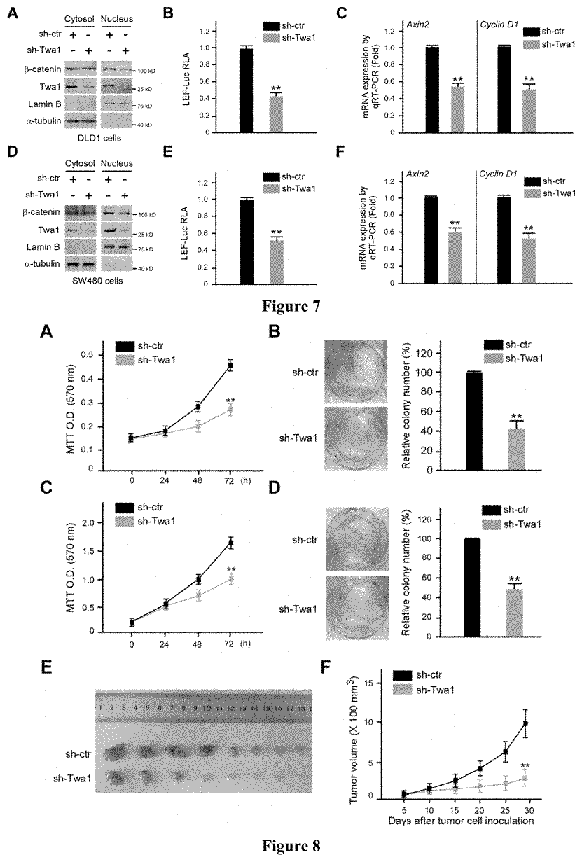

Aberrant activation of Wnt signaling pathway is one of the major causes of colorectal cancer. Since Twa1 was highly expressed in the colorectal cancer tissues and participated in the regulation of Wnt signaling pathway, the inventors further investigated whether Twa1 participated in this pathological process by regulating the canonical Wnt signaling pathway. In The colorectal cancer cell lines DLD1 and SW480, β-catenin is constitutively highly expressed in the nucleus due to mutation of the APC gene. In these cell lines, Twa1 shRNA lentivirus efficiently decreased Twa1 expression, and then cytoplasmic and nuclear fraction were extracted. As shown in FIGS. 7A and D, depletion of Twa1 significantly reduced the nuclear level of β-catenin in DLD1 and SW480 cells. Further dual luciferase reporter gene assay and real-time quantitative PCR assay showed that knockdown of Twa1 inhibits the activity of Wnt reporter genes and Wnt target genes Axin2 and Cyclin D1 expression in DLD1 and SW480, respectively (FIGS. 7B, C, E, and F). These results indicate that in colorectal cancer cell lines, Twa1 is also involved in the regulation of β-catenin nuclear accumulation in Wnt signaling.

### Embodiment 8, Twa1 Promotes the Growth and Enhances the Tumorigenicity of Colorectal Cancer Cells

8.1 Since the canonical Wnt signaling pathway is associated with the cancer biological characteristics of colorectal cancer cells, the inventors further examined the effect of down-regulation of Twa1 on the biological behavior of colorectal cancer cells. The results of the MTT assay were shown in FIGS. 8A and C. Knockdown of Twa1 significantly inhibited the proliferation of DLD1 cells and SW480 cells. The ordinate showed the value of the absorbance, which was proportional to the number of cells. Colony formation assay displayed that depletion of Twa1 significantly inhibited the colony formation of DLD1 cells and SW480 cells (FIGS. 8B and D). The ordinate showed the relative clone formation rate. These results indicate that Twa1 promotes proliferation of colorectal cancer cells in vitro. The detailed methods of MTT assay and plate colony formation assay are shown as follows:

8.1.1 Mtt Assay:

(1) Collected the cells in the exponential growth phase, adjusted the cell suspension concentration, added 100 μl/well cell suspensions to the 96-well plate, and made the cell density to be measured at 1000-5000/well.

(2) The culture plate was placed in an incubator and the culture time was determined according to the experimental requirements.

(3) 20 μl MTT solution (5 mg/ml, 0.5% MTT) was added to each well, and incubated for 4 hours.

(4) After MTT reaction, carefully removed the culture medium from the well. 150 μl DMSO solution was added to each well, and shaken on a shaker at room temperature for 10 minutes at room temperature to dissolve the crystals sufficiently.

(5) The absorbance of each well was measured at the OD value of 570 nm in a microplate reader.

8.1.2 Colony Formation Experiment

(1) Each group of cells in the exponential growth phase was digested with 0.25% trypsin and pipetted to individual cells, and the cells were suspended in RPMI-1640 medium containing 10% serum for use.

(2) Diluted the cell suspension as a gradient. Each group of cells was seeded onto a 6-well plate containing 2 ml of culture solution at a gradient density of 50, 100, and 200 cells per dish, and gently shaken to evenly disperse the cells. Incubated for 2 to 3 weeks, and replaced with fresh medium several times.

(3) When clones appeared in the culture dish, the culture was terminated. The cell culture medium was discarded and cells were carefully washed twice with 1×PBS, and then fixed with 4% paraformaldehyde for 15 minutes. Subsequently, the fixing solution was discarded, and the crystal violet staining solution was added for 5 minutes. Finally, the staining solution was slowly washed away with running water and air-dried.

(4) Inverted the plate, directly counted the number of clones with the naked eye, or counted the number of clones larger than 10 cells in a microscope. Finally, the clone formation rate was calculated.

Clonal formation rate=(number of clones/number of cells inoculated)×100%

8.2 The effect of Twa1 on proliferation and tumorigenesis of colorectal cancer cells in vivo was examined by a xenograft mouse model. Wild-type DLD1 cells and DLD1 cells depleted of Twa1 expression were inoculated subcutaneously in nude mice, and the size of subcutaneous tumor growth in nude mice was observed and measured. As shown in FIG. 8E, knockdown of Twa1 significantly inhibited the growth of tumor, indicating that Twa1 is essential for the proliferation and tumorigenicity of colorectal cancer cells in vivo. FIG. 8F showed the growth curve of the tumor in nude mice. The abscissa showed the number of days after tumor inoculation and the ordinate showed tumor volume. The experiment procedures of tumor formation in nude mice are as follows:

The cells were digested with trypsin and made into a cell suspension, and after washing three times with serum-free medium, the cells were counted, and the cell density was adjusted to be 1×107 cells/ml. A suspension of 0.2 ml (containing 2×106 tumor cells) was injected subcutaneously into nude mice for 28 days. The length and width of the tumor were measured weekly using a vernier caliper, and the tumor volume was calculated.

Calculated the tumor volume formula as V=½×L×W2 (V: volume; L: long; W: wide)

### Embodiment 9, the Nuclear Expression Level of Twa1 was Associated with the Prognosis of Colorectal Cancer Patients

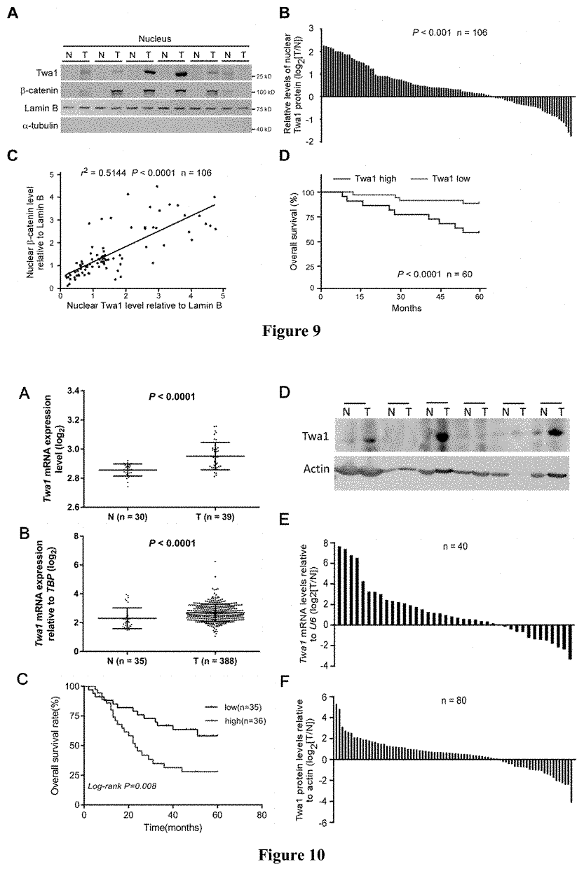

To illustrate the clinical significance of Twa1 expression in the nucleus, 106 pairs of colorectal cancer tissues and their nontumor tissues were collected. The nuclear fractions of these tissues were extracted and the expression of Twa1 and β-catenin was detected by immunoblotting. As shown in FIG. 9A, Twa1 was up-regulated in the colorectal cancer tissues (P<0.001). FIG. 9B showed a quantitative statistical analysis of the panel A, and the intensities of the Twa1 blots were quantified and normalized by Image J software (NIH, National Institutes of Health). The ordinate indicated the expression level of the Twa1 protein relative to the internal reference lamin B protein after log 2 transformation. P<0.01, Student's t test. FIG. 9C showed that the level of Twa1 expression in the nucleus was positively correlated with the expression level of β-catenin in the nucleus (r2=0.5144, P<0.0001). More importantly, according to the median expression level of Twa1 in 60 patients with colorectal cancer, the patients were divided into a group with high Twa1 expression level and a low Twa1 expression level. Statistical analysis in FIG. 9D showed that colorectal cancer patients with high Twa1 expression had a lower five-year survival rate (P<0.0001). The abscissa showed the survival time, and the ordinate showed the proportion of survivors.

### Embodiment 10, Twa1 Expression was Significantly Up-Regulated in Human Gastric Cancer Tissues

10.1 To illustrate the expression level of Twa1 in gastric cancer, RNA sequencing data in gastric cancer tissues in the Oncomine database were analyzed. A total of 39 gastric cancer tissue samples and 30 nontumor tissue samples were included in the data set. FIG. 10A showed that the Twa1 mRNA level was up-regulated in gastric cancer tissues. RNA sequencing data for gastric cancer tissues in the TCGA (The Cancer Genome Atlas) database was further analyzed. A total of 388 gastric cancer tissue samples and 35 nontumor tissue samples were included in the database. FIG. 10B showed that Twa1 mRNA was up-regulated in gastric cancer tissues. Each point in the figure represented the expression level of the Twa1 gene relative to the internal reference gene TBP (TATA binding protein) after log 2 transformation. T indicated tumor tissue and N indicated the matched nontumor tissues. The ordinate indicated the expression level of the Twa1 gene relative to the internal reference gene TBP after log 2 transformation. The black horizontal line shows the median±standard deviation. P<0.0001, Student's t test.

10.2 To illustrate the clinical significance of Twa1 expression in gastric cancer, the clinical data of GSE57303 database of gastric cancer were analyzed. A total of 71 pairs of gastric cancer tissue samples and their adjacent tissue samples were included in the database. Statistical analysis showed that gastric cancer patients with high levels of Twa1 had a lower five-year survival rate (P<0.0001). In FIG. 10C, the abscissa showed the survival time, and the ordinate showed the proportion of survivors.

10.3 Further extracted the total protein of gastric cancer tissues and nontumor tissues, and detected the protein expression level of Twa1 in gastric cancer tissues. The samples were obtained from the Zhejiang Cancer Hospital mentioned. As shown in FIG. 10D, Twa1 was up-regulated in the protein level of gastric cancer.

### Embodiment 11, Knockout of Twa1 Inhibited the Migration and Invasion of Gastric Cancer Cells

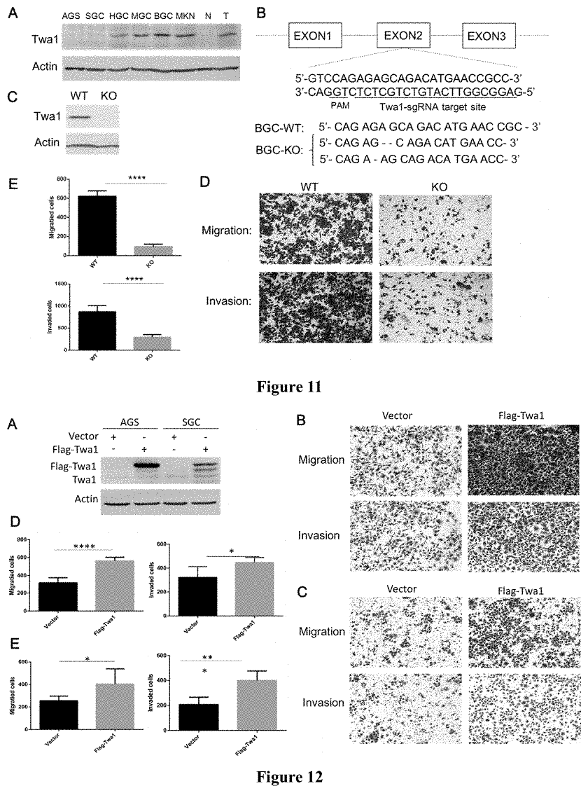

11.1 In order to investigate the role of Twa1 in BGC cell lines highly expressing Twa1, the Twa1 gene was knocked out by using the CRISPR/Cas9 system. FIG. 11A shows sequencing results of the Twa1 gene in normal BGC cells, and the sequencing results of the Twa1 gene edited by the CRISPR/Cas9 system in Twa1 knockout BGC cells. In Twa1-KO cells, the protein expression of the edited Twa1 gene was prematurely terminated, and the Twa1 protein was successfully knocked out. FIG. 11B showed that knockout of the Twa1 gene completely inhibited Twa1 protein expression.

11.2 To further illustrate the effect of down-regulation of Twa1 expression on the biological behavior of gastric cancer. Transwell assay in FIG. 11D revealed that knockdown of Twa1 significantly inhibited BGC cell migration and invasion compared to the control group. The Transwell experimental steps are as follows:

11.2.1 Transwell chamber preparation. Invasion experiments required Matrigel, and migration experiments did not require Matrigel.

(1) Diluted Matrigel with serum-free medium.

(2) Took 50 μl diluted Matrigel and spread it evenly on the bottom of the chamber.

(3) Placed the Matrigel-packed chamber in a 24-well plate and place the 24-well plate in a cell culture incubator for 30 min.

11.2.2 Preparation of Cell Suspension

(1) Before preparing the cell suspension, the cells could be serum-starved for 12-24 hours.

(2) Digested the cells, terminated the digestion, discarded the culture medium by centrifugation, washed 1-2 times with PBS, cells were resuspended in serum-free medium. Adjusted the cell density to 250,000/mL.

11.2.3 Inoculation of Cells

(1) 200 μl the cell suspension was added to the Transwell chamber.

(2) 500 μl medium containing 10% serum was added to a 24-well plate.

(3) The chamber was gently placed in a 24-well plate, and the 24-well plate was placed in a cell culture incubator for 24-48 hours.

11.2.4 Statistics Analysis

(1) Took out the chamber, added 500 μl 4% PFA to the wells of the 24-well plate, gently placed the chamber and fixed it in PFA for more than 30 minutes.

(2) The chamber was taken out, and 500 μl 0.1% crystal violet was added to the other wells of the 24-well plate, and stained for 10-30 mim.

(3) Washed the chamber with PBS, and then gently wiped off the Matrigel and the cells in the upper chamber with a cotton swab.

(4) Observed and photographed with an inverted microscope, took 6 images for each chamber, and counted the cells.

### Embodiment 12, Overexpression of Twa1 Enhanced Migration and Invasion of Gastric Cancer Cells

To further validate the effect of Twa1 expression on the biological behavior of gastric cancer. Twa1 was overexpressed in Twa1 low-expressing AGS and SGC cells, and FIG. 12A showed that Twa1 protein was overexpressed in AGS and SGC cells transfected with Twa1 plasmids. FIGS. 12B and C showed that ectopic expression of Twa1 enhanced the migration and invasion of AGS cells and SGC cells.

### Embodiment 13, Knockout of Twa1 Promoted Mesenchymal-Epithelial Transition (MET) in Gastric Cancer Cells

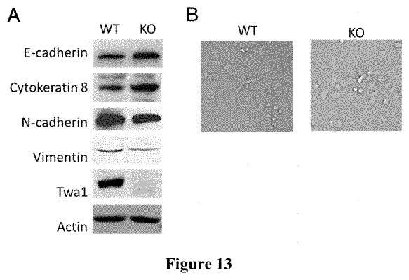

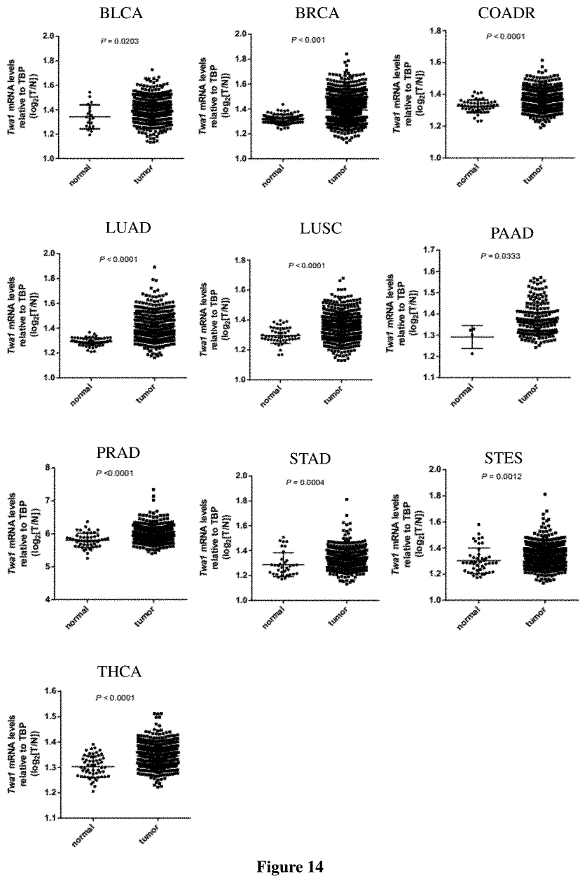

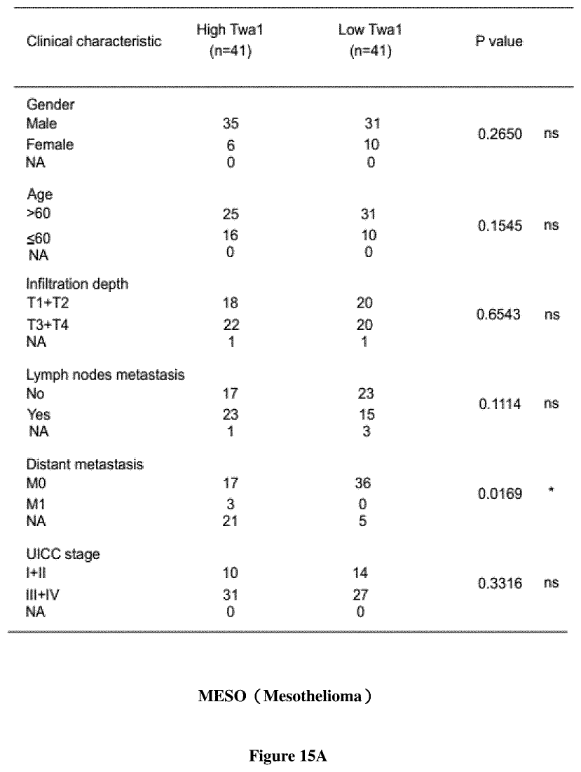

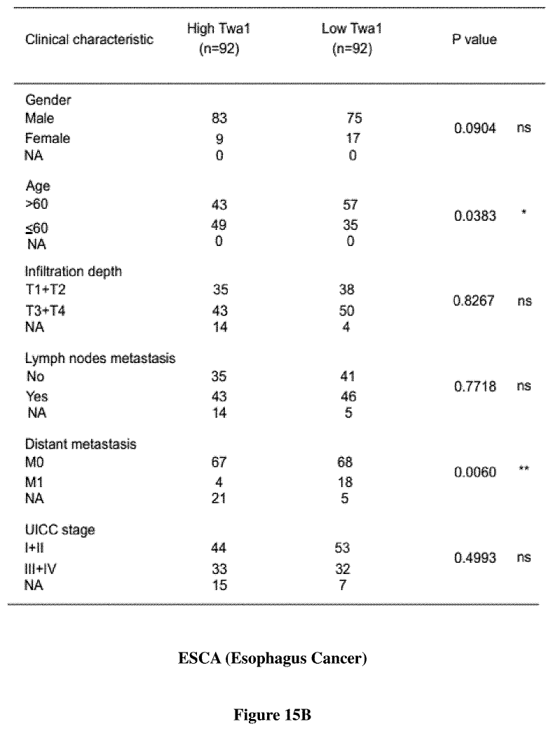

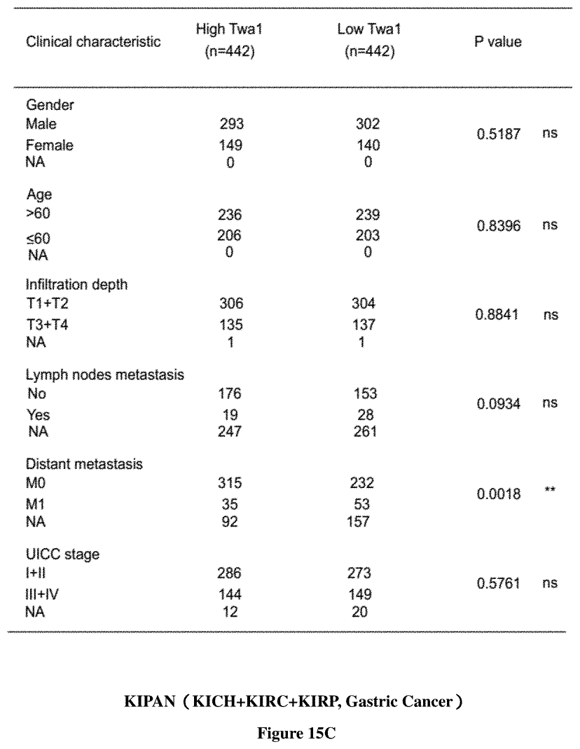

To further elucidate the effect of knockout of Twa1 on the migration and invasion of gastric cancer cells, the expression levels of epithelial marker proteins and mesenchymal cell marker proteins were examined by western blotting in wild-type BGC cells and Twa1-KO BGC cells. As shown in FIG. 13, knockout of Twa1 upregulated the expression level of epithelial cell marker proteins (E-cadherin and Cytokeratin 8), and downregulated the expression level of mesenchymal cell marker proteins (N-cadherin and Vimentin). These data suggest that knockout of Twa1 promotes mesenchymal-epithelial transition (MET) in gastric cancer cells.

### Embodiment 14, Twa1 is Upregulated in a Variety of Common Human Malignant Tumor Tissues

In order to further explore the expression level of Twa1 in human malignant tumor tissues, the expression level of Twa1 mRNA in various tumors was analyzed by bioinformatics method. The results showed that Twa1 was up-regulated in 10 tumors, including bladder cancer, breast cancer, colorectal cancer, lung adenocarcinoma, lung squamous cell carcinoma, pancreatic cancer, prostate cancer, stomach cancer, bronchial adenoma, and thyroid cancer.

### Embodiment 15, the Expression Level of Twa1 was Associated with Metastasis of a Variety of Human Malignancies

To further investigate the relationship between the expression level of Twa1 and the metastasis of common malignant tumors in humans, bioinformatics analysis was used to analyze the correlation between Twa1 mRNA expression levels and pathological features of patients. Taken pleural cancer, esophageal cancer and renal cancer as examples, the results showed that the high expression of Twa1 was associated with tumor metastasis (P<0.05).

The above embodiments demonstrate that Twa1 plays an important role in the proliferation, growth and metastasis of tumor cells. In colorectal cancer, knockdown of Twa1 inhibits cell proliferation. In gastric cancer, overexpression of Twa1 enhances cell migration and invasion, and knockout of Twa1 in gastric cancer cells inhibits cell migration and invasion. Twa1 promotes cell proliferation by enhancing the accumulation of β-catenin in the nucleus, and the accumulation of β-catenin nucleus is often observed in tumors. Therefore, Twa1 promotes the proliferation of tumor cells, which has been verified in colorectal cancer cells. In breast cancer, sarcoma, lung cancer, prostate cancer, kidney cancer, pancreatic cancer, blood cancer, neuroblastoma, glioma, head cancer, neck cancer, thyroid cancer, liver cancer, ovarian cancer, vulvar cancer, cervical cancer, endometrial cancer, testicular cancer, bladder cancer, esophageal cancer, gastric cancer, nasopharyngeal carcinoma, buccal cancer, oral cancer, gastrointestinal stromal tumor, skin cancer, multiple myeloma, glioblastoma, and melanoma, Twa1 is also up-regulated in various tumors, suggesting that Twa1 may have a role in promoting tumor cell proliferation in these tumors. EMT occurs in the development of many tumors, and Twa1 promotes cell migration and invasion by regulating epithelial-mesenchymal transition (EMT) of cells, which has already been verified in gastric cancer cells. In breast cancer, sarcoma, lung cancer, prostate cancer, kidney cancer, pancreatic cancer, blood cancer, neuroblastoma, glioma, head cancer, neck cancer, thyroid cancer, liver cancer, ovarian cancer, vulva cancer, cervical cancer, endometrial cancer, testicular cancer, bladder cancer, esophageal cancer, gastric cancer, nasopharyngeal cancer, buccal cancer, oral cancer, gastrointestinal stromal tumor, skin cancer, multiple myeloma, glioblastoma and melanoma, Twa1 is up-regulated in various tumors, and the expression level is correlated to tumor metastasis, suggesting that Twa1 may also have the ability to promote tumor cell migration and invasion in these tumors.

In conclusion, the expression level of Twa1 is associated with the occurrence and development of tumors and is a potential diagnostic and therapeutic target. Therefore, using the Twa1 gene and the expression product according to the present disclosure as a target, the tumor detection kit could be designed and developed, a drug screening model could be established, and the anti-tumor drug could be screened and prepared, which provides important theoretical and practical value for inhibiting or interfering with the occurrence and development of tumors.

What is claimed is:

1. An antibody, prepared by using a polypeptide or protein as an antigen or based on a sequence of the polypeptide or protein, wherein the polypeptide or protein comprises at least one of the following characteristics:
1) the amino acid sequence of SEQ ID NO. 2;
2) a protein which has been subjected to substitution and/or deletion and/or addition of one or several amino acid residues in the amino acid residue sequence of SEQ ID NO. 2, and associated with tumor; and
3) a polypeptide or protein having 90% or more homology with the amino acid residue sequence of SEQ ID NO. 2, and associated with tumor.

2. The antibody according to claim 1, wherein the polypeptide or protein is produced by artificial synthesis or by expression and purification from a transgenic cell line or host strain containing a Twa1 expression vector; the Twa1 expression vector contains DNA sequence of the Twa1 gene.

3. The antibody according to claim 1, wherein the substitution and/or deletion and/or addition of one or several amino acid residues includes substitution and/or deletion and/or addition of no more than 10 amino acid residues.

4. The antibody according to claim 1, wherein the amino acid sequence of SEQ ID NO. 2 consists of 228 amino acid residues.

5. The antibody according to claim 1, wherein the antibody is selected from the monoclonal antibodies, polyclonal antibodies, chimeric antibodies, single chain antibodies, humanized antibodies, Fab fragments, and products of Fab expression libraries.

6. The antibody according to claim 1, wherein the tumor is selected from colorectal cancer, breast cancer, sarcoma, lung cancer, prostate cancer, renal cancer, pancreatic cancer, blood cancer, neuroblastoma, glioma, head cancer, neck cancer, thyroid cancer, liver cancer, ovarian cancer, vulvar cancer, cervical cancer, endometrial cancer, testicular cancer, bladder cancer, esophageal cancer, stomach cancer, nasopharyngeal cancer, buccal cancer, oral cavity cancer, gastrointestinal stromal tumors, skin cancer, multiple myeloma, glioblastoma, and melanoma.

7. The antibody according to claim 1, wherein when the antibody is applied in the clinic, the antibody indicates whether the patient providing the sample has a tumor; when a high amount of Twa1 polypeptide or protein in the clinical sample is detected, the patient who provide the clinical sample is likely to have a tumor; when a low amount of Twa1 polypeptide or protein in the clinical sample is detected, the patient who provide the clinical sample is likely not to have a tumor.

8. A protein complex, wherein the protein complex comprises the β-catenin protein and the protein or polypeptide of claim 1, the protein or polypeptide promotes β-catenin nuclear accumulation, thereby further promoting tumor cell proliferation.

9. The protein complex according to claim 8, wherein the polypeptide or protein is produced by artificial synthesis or by expression and purification from a transgenic cell line or host strain containing a Twa1 expression vector; the Twa1 expression vector contains DNA sequence of the Twa1 gene.

10. The protein complex according to claim 8, wherein the substitution and/or deletion and/or addition of one or several amino acid residues includes substitution and/or deletion and/or addition of no more than 10 amino acid residues.

11. The protein complex according to claim 8, wherein the amino acid sequence of SEQ ID NO. 2 consists of 228 amino acid residues.

12. The protein complex according to claim 8, wherein the tumor is selected from colorectal cancer, breast cancer, sarcoma, lung cancer, prostate cancer, renal cancer, pancreatic cancer, blood cancer, neuroblastoma, glioma, head cancer, neck cancer, thyroid cancer, liver cancer, ovarian cancer, vulvar cancer, cervical cancer, endometrial cancer, testicular cancer, bladder cancer, esophageal cancer, stomach cancer, nasopharyngeal cancer, buccal cancer, oral cavity cancer, gastrointestinal stromal tumors, skin cancer, multiple myeloma, glioblastoma, and melanoma.

13. An anti-tumor drug, wherein the anti-tumor drug comprises the antibody of claim 1, the protein complex of claim 8, and at least one pharmaceutically acceptable carrier or excipient.

14. The anti-tumor drug according to claim 13, wherein the anti-tumor drug is selected from a cytotoxic drug, a hormonal drug, a biological response modifier, an antibody drug, a cell differentiation inducer, an apoptosis inducer, a neovascularization inhibitor, an epidermal growth factor receptor inhibitor, a gene therapy drug, a tumor vaccine.

15. The anti-tumor drug according to claim 14, wherein the anti-tumor drug is selected from the anti-tumor small molecule drugs and anti-tumor small molecule drug compositions.

16. The anti-tumor drug according to claim 13, wherein the substitution and/or deletion and/or addition of one or several amino acid residues includes substitution and/or deletion and/or addition of no more than 10 amino acid residues.

17. The anti-tumor drug according to claim 13, wherein the amino acid sequence of SEQ ID NO. 2 consists of 228 amino acid residues.

18. The anti-tumor drug according to claim 13, wherein the tumor is selected from colorectal cancer, breast cancer, sarcoma, lung cancer, prostate cancer, renal cancer, pancreatic cancer, blood cancer, neuroblastoma, glioma, head cancer, neck cancer, thyroid cancer, liver cancer, ovarian cancer, vulvar cancer, cervical cancer, endometrial cancer, testicular cancer, bladder cancer, esophageal cancer, stomach cancer, nasopharyngeal cancer, buccal cancer, oral cavity cancer, gastrointestinal stromal tumors, skin cancer, multiple myeloma, glioblastoma, and melanoma.

19. The anti-tumor drug according to claim 13, wherein the pharmaceutically acceptable carrier comprises diluents, fillers, binders, wetting agents, disintegrating agents, absorption enhancers, surfactants, adsorption carriers, lubricants.

20. The anti-tumor drug according to claim 13, wherein the anti-tumor drug is made into one or more forms selecting from tablets, powders, granules, capsules, oral liquids and injections.
